(19) United States
(12) Patent Application Publication
(10) Pub. No.: US 2022/0023250 A1
(43) Pub. Date: Jan. 27, 2022

(54) ISOPROPYLCARBONATE BENZOYL PEROXIDE COMPOSITIONS AND METHODS OF USE

(71) Applicant: Galderma Holding SA, La Tour-de-Peilz (CH)

(72) Inventors: Karine Nadau-Fourcade, Villeneuve-Loubet (FR); Jean-Christophe Buge, Nice (FR)

(73) Assignee: Galderma Holding SA, La Tour-de-Peilz (CH)

(21) Appl. No.: 17/492,320

(22) Filed: Oct. 1, 2021

Related U.S. Application Data
(63) Continuation of application No. PCT/IB2020/053223, filed on Apr. 3, 2020.
(60) Provisional application No. 62/829,507, filed on Apr. 4, 2019.

(51) Int. Cl. A61K 31/327; A61P 17/10; A61K 47/02; A61K 47/10; A61K 47/20; A61K 47/32; A61K 47/26; A61K 47/18

(52) U.S. Cl. CPC A61K 31/327; A61P 17/10; A61K 47/02; A61K 47/183; A61K 47/20; A61K 47/32; A61K 47/26; A61K 47/10

(57) ABSTRACT

Described herein are compositions comprising (i) Isopropylcarbonate Benzoyl Peroxide, (ii) an opacifier, and (iii) and a preserving agent that have good physical, chemical and microbiological stability and corresponding methods of use.

### CROSS-REFERENCE TO RELATED PATENT APPLICATIONS

This application is a continuation of PCT/IB2020/053223, filed Apr. 3, 2020, which claims priority to U.S. Provisional Patent Application No. 62/829,507, filed Apr. 4, 2019, the entire disclosures of which is hereby incorporated by reference in their entireties for any and all purposes.

### FIELD

Described herein are compositions comprising Isopropylcarbonate Benzoyl Peroxide that have good physical, chemical, and microbiological stability and corresponding methods of use.

### BACKGROUND

The following discussion is provided to aid the reader in understanding the invention disclosed and is not admitted constitute prior art thereto.

Acne vulgaris is a chronic disorder of the pilosebaceous follicles (apparati) which is characterized by comedones (blackheads), papules, pustules, cysts, nodules and often scars which appear in the most visible regions of the skin, in particular the face, chest, back and sometimes the neck and top of the arms. Acne is a condition comprising several stages and, in its severest form, results in the hospitalization of the patient and significant discomfort with the long-term presence of skin scars.

Many treatments are currently available for treating acne but each treatment unfortunately has limits which would be desirable to overcome. For instance, oral administration of anti-acne agents is commonly provided in severe cases of acne. However, numerous side effects have been associated with the oral administration of antiacne active compounds. Specifically, isotretinoin, which is a vitamin A derivative, exhibits associated risks of teratogenicity and it can constitute a risk to women of reproductive age. The oral administration of antibiotics suitable for the treatment of acne can also be accompanied by side effects, such as abdominal cramps, coughing, diarrhea, fatigue, buccal irritation and other undesirable symptoms. Topical anti-acne medication, such as retinoids, are associated with elevated skin irritation, and thus, careful consideration must be given to the tolerability of a potential maintenance therapy.

There exists a need for improved treatments of acne which effectively prevent the condition from evolving towards its severest form and which can be used without unfavorable effects by the majority of the people affected.

Isopropylcarbonate Benzoyl Peroxide is a peroxide derivative that has demonstrated antiacne effectiveness. Formulating Isopropylcarbonate Benzoyl Peroxide into dermatological or cosmetic products is challenging as Isopropylcarbonate Benzoyl Peroxide is an unstable compound that releases benzoic acid and salicylic acid upon contact with skin. Similar to benzoyl peroxide, the efficacy of Isopropylcarbonate Benzoyl Peroxide is associated with its decomposition when it is placed in contact with the skin. Specifically, the oxidizing properties of the free radicals produced during this decomposition lead to the desired effect. Thus, in order to maintain the optimum efficacy of Isopropylcarbonate Benzoyl Peroxide, it is important to prevent its decomposition before use, i.e., during storage.

Thus, there exists a clear medical and cosmetic need for the treatment of acne-related conditions and pathologies. The present invention satisfies this need by providing Isopropylcarbonate Benzoyl Peroxide compositions that are useful for treating acne, where the compositions have good physical, chemical and microbiological stability.

### SUMMARY

Provided in one aspect is a topically applicable composition comprising:

i) Isopropylcarbonate Benzoyl Peroxide;

ii) an opacifier; and

iii) a preserving agent.

In some embodiments, the composition comprises about 0.0001% to about 20%, about 0.001% to about 20%, about 0.01% to about 20%, about 0.1% to about 20%, about 0.1% to about 10%, about 0.1% to about 5%, about 2.5% to about 5% by weight of Isopropylcarbonate Benzoyl Peroxide. In some embodiments, the composition comprises about 3% by weight of Isopropylcarbonate Benzoyl Peroxide.

In some embodiments, the opacifier is bismuth oxychloride, titanium dioxide, fluorphlogopite, mica, iron oxide, nylon polymer, polymethyl methacrylate, boron nitride, kaolin, glycol distearate, styrene copolymer, a fatty alcohol, or any combination thereof. In some embodiments, the opacifier is bismuth oxychloride, titanium dioxide, fluorphlogopite, mica, iron oxide, nylon polymer, polymethyl methacrylate, boron nitride, or any combination thereof. In some embodiments, the opacifier is titanium dioxide. In some embodiments, the titanium dioxide is pharmaceutical grade or cosmetic grade. In some embodiments, the composition comprises about 0.1% to about 2.5% by weight of the opacifier. In some embodiments, the composition comprises about 0.4% by weight of the opacifier.

In some embodiments, the preserving agent is phenoxyethanol, potassium sorbate, benzyl alcohol, methyl paraben, chlorhexidine digluconate, chloroxylenol, chlorphenesin, dehydroacetic acid, diazolidinyl urea, DMDM hydantoin, ethylparaben, iodopropynyl butylcarbamate, methylisothiazolinone, propyllparaben, phenoxyethanol, phenoxyisopropanol, polyaminopropyl biguianide, sodium benzoate, salicylic acid, or any combination thereof. In some embodiments, the preserving agent is phenoxyethanol, potassium sorbate, benzyl alcohol, methyl paraben, or any combination thereof. In some embodiments, the preserving agent is phenoxyethanol. In some embodiments, the composition comprises about 0.1% to about 0.8% by weight of the preserving agent. In some embodiments, the composition comprises about 0.4% or about 0.8% by weight of the preserving agent.

In some embodiments, the composition further comprises a surfactant. In some embodiments, the surfactant is docusate sodium (diethylhexyl sodium sulfosuccinate), poloxamer, PEG ether, PPG ester, alkyl polyglucoside, sorbitan ester, ethoxylated sorbitan ester, alcohol sulfate, ethoxylated alcohol sulfate, alkyl benzene sulfonate, alpha olefin sulfonate, sulfosuccinate, isethionate ester, taurate, alkyl betaine, alkyl amidopropyl betaine, amphoacetate, or alkyl sultaine. In some embodiments, the surfactant is docusate sodium (diethylhexyl sodium sulfosuccinate). In some embodiments, the composition comprises about 0.05% by weight of the surfactant.

In some embodiments, the composition further comprises a humectant. In some embodiments, the humectant is glycerol. In some embodiments, the composition comprises about 4% by weight of the humectant.

In some embodiments, the composition further comprises a liquid wetting surfactant. In some embodiments, the liquid wetting surfactant is a poloxamer. In some embodiments, the liquid wetting surfactant is poloxamer 124. In some embodiments, the composition comprises about 0.2% by weight of the liquid wetting surfactant.

In some embodiments, the composition further comprises a gelling agent. In some embodiments, the gelling agent comprises acrylates/Steareth-20 methacrylate copolymer, acrylamide/sodium acrylate copolymer, acrylamide/sodium acryloyl dimethyltaurate copolymer/isohexadecane/polysorbate-20, acrylamide/ammonium acrylate copolymer, polyisobutene, polysorbate 20, acrylamidopropyltrimonium chloride/acrylamide copolymer, acrylates copolymer, acrylates/C10-30 alkyl acrylates crosspolymer, acrylates/acrylamide copolymer and mineral oil and polysorbate-85, acrylates/C12-22 alkyl methacrylate copolymer, acrylates/vinyl ssodecanoate croospolymer, acrylic acid copolymer, ammomium acryloyldimethyltaurate/beheneth-25 methacrylate copolymer, ammomium acryloyldimethyltaurate/VP copolymer, ammonium polyacrylate/isohexadecane/PEG 40 castor oil/sorbitan oleate/aqua, biosaccharide gum-1, bentonite/xanthan gum (smectite), C13-14 isoparafin/mineral oil/sodium polyacrylate/polyacrylamide/polysorbate 85 carbomer, carboxyvinyl polymer, cellulose gum, glyceryl polymethacrylate, hydrogenated polydecene, ethylene/propylene/styrene copolymer, butylene/ethylene/styrene copolymer, hydroxyethyl acrylate/sodium acryloyldimethyltaurate copolymer/squalane/polysorbate 60/water, hydroxyethylcellulose, hydroxypropyl starch phosphate, hydroxypropylcelulose, magnesium aluminium silicate, magnesium silicate, methyl vinyl ether/maleic anhydride (PVM/MA decadiene crosspolymer)), polyacrylamide/C13-14 Isoparafin/Laureth-7/aqua, polyacrylate 13/polyisobutene/polysorbate 20, potato starch modified, propane-1,2-diol alginate, PVP (polyvinylpyrrolidone), sclerotium gum, sodium acrylate copolymer/PPG-1 trideceth 6/parafinum liquidum/sorbitan trioleate/aqua, sodium acrylate/acryloyldimethyltaurate/copolymer & isohexadecane & polysorbate, sodium acrylate/acryloyldimethyltaurate, copolymer/polyisobutene/caprilyl capryl glucoside, sodium acrylates copolymer/glycine soja/PPG-1 trideceth-6 (anionic), sodium acrylates copolymer/parafinium liquidum/PPG-1 trideceth-6 (anionic), sodium acrylates copolymer/hydrogenated polyisobutene/phospholipids/polyglyceryl-10 stearate/Helianthus annuus seed oil, sodium carbomer, sodium polyacrylate, sodium polyacrylate/hydrogenated polydecane, steareth-10 allyl ether/acrylates copolymer, succinoglycan, water/glycerin/polyacrylimidomethylpropane/sulfonate/polyquaternium-4, xanthan gum, xanthan gum/magnesium aluminium silicate, xanthan gum/hectorite/cellulose, and/or magnesium aluminium silicate. In some embodiments, the gelling agent comprises acrylamide, sodium acryloyldimethyl taurate copolymer, isohexadecane and polysorbate 80 (SIMULGEL™ 600). In some embodiments, the gelling agent comprises hydroxyethyl acrylate, sodium acryloyldimethyl taurate copolymer, isohexadecane and polysorbate 60 (SIMULGEL™ INS 100). In some embodiments, the gelling agent comprises hydroxyethyl acrylate and sodium acryloyldimethyl taurate copolymer (SEPINOV™ EMT10). In some embodiments, the gelling agent is pharmaceutical grade or cosmetic grade. In some embodiments, the composition comprises about 0.1% to about 10% by weight of the gelling agent. In some embodiments, the composition comprises about 4% by weight of the gelling agent.

In some embodiments, the composition further comprises a pro-penetrating agent. In some embodiments, the pro-penetrating agent is propylene glycol, dipropylene glycol, propylene glycol dipelargonate, lauroglycol, an alcohol, an alkylmethyl sulfoxide, a polyol, oleic acid, isopropyl myristate, decylmethyl sulfoxide, DMSO, urea, or ethoxydiglycol. In some embodiments, the pro-penetrating agent is propylene glycol, dipropylene glycol, propylene glycol dipelargonate, lauroglycol, or ethoxydiglycol. In some embodiments, the pro-penetrating agent is propylene glycol. In some embodiments, the composition comprises about 4% by weight of the pro-penetrating agent.

In some embodiments, the composition further comprises a sequestering agent. In some embodiments, the sequestering agent is disodium ethylenediaminetetraacetic acid (EDTA), dihydroxyethylglycine, trisodium ethylenediamine fisuccinate glutamic acid diacetic acid tetra sodium salt (GLDA), fiethylenetriaminepentaacetic acid (DTPA), methylglycindiacetic acid (MGDA), hydroxyethylethylenediaminetriacetic acid (HEDTA), glucoheptonate, nitrilotriacetic acid (NTA), ethylenediaminedisuccinic acid (EDDS), iminodisuccinic acid (IDS), ethylenediamine-N,N′-diglutaric acid (EDDG), ethylenediamine-N,N′-dimalonic acid (EDDM), 3-hydroxy-2,2-iminodisuccinic acid (HIDS), 2-hydroxyethyliminodiacetic acid (HEIDA), pyridine-2,6-dicarboxylic acid (PDA), etidronic acid. carnosine, phytic acid, kojic acid, sodium carboxymethyl inulin, citric acid, tartaric acid or a derivative or salt thereof. In some embodiments, the sequestering agent is disodium ethylenediaminetetraacetic acid (EDTA), dihydroxyethylglycine, citric acid, tartaric acid or a derivative or salt thereof. In some embodiments, the sequestering agent is disodium ethylenediaminetetraacetic acid (EDTA). In some embodiments, the composition comprises about 0.1% to about 0.5% by weight of the sequestering agent. In some embodiments, the composition comprises about 0.1% or about 0.2% by weight of the sequestering agent.

In some embodiments, the composition further comprises a vehicle. In some embodiments, the vehicle is purified water. In some embodiments, the composition is further formulated as a gel. In some embodiments, the composition is further formulated as a cream.

In some embodiments, the composition further comprises a keratolytic agent, an oil, an antioxidant, a skin conditioning agent, or any combination thereof. In some embodiments, the keratolytic agent is lactic acid, glycolic acid, malic acid, phytic acid, silver or a silver solution. In some embodiments, the oil comprises mineral oil, hydrogenated polyisobutene, squalene, polydecene, or any combination thereof. In some embodiments, the antioxidant comprises butylated hydroxytoluene, DL alpha tocopherol, ascorbyl palmitate, or any combination thereof. In some embodiments, the skin conditioning agent comprises silica beads, methyl methacrylate cross polymer, nylon polymer, mica, polymethyl methacrylate, titanium dioxide, or any combination thereof.

Also provided in one aspect is a topically applicable composition comprising:

i) Isopropylcarbonate Benzoyl Peroxide;

ii) titanium dioxide as an opacifier;

iii) phenoxyethanol as a preserving agent;

iv) docusate sodium (diethylhexyl sodium sulfosuccinate) as a surfactant;

v) glycerol as a humectant;

vi) poloxamer 124 as a liquid wetting surfactant;

vii) a combination of acrylamide, sodium acryloyldimethyl taurate copolymer, isohexadecane, and polysorbate 80 as a gelling agent;

viii) propylene glycol as a pro-penetrating agent;

ix) disodium EDTA as a sequestering agent; and

x) purified water as a vehicle.

Also provided in one aspect is a topically applicable composition comprising:

i) about 3% by weight of Isopropylcarbonate Benzoyl Peroxide;

ii) about 0.4% by weight of titanium dioxide as an opacifier;

iii) about 0.4% by weight of phenoxyethanol as a preserving agent;

iv) about 0.05% by weight of docusate sodium (diethylhexyl sodium sulfosuccinate) as a surfactant;

v) about 4% by weight of glycerol as a humectant;

vi) about 0.2% by weight of poloxamer 124 as a liquid wetting surfactant;

vii) about 4% by weight of acrylamide, sodium acryloyldimethyl taurate copolymer, isohexadecane and polysorbate 80 as a gelling agent

viii) about 4% by weight of propylene glycol as a pro-penetrating agent;

ix) about 0.1% or about 0.2% by weight of disodium EDTA as a sequestering agent; and

x) purified water as a vehicle.

Also provided in one aspect is a topically applicable composition comprising:

i) about 3% by weight of Isopropylcarbonate Benzoyl Peroxide;

ii) about 0.4% by weight of titanium dioxide as an opacifier;

iii) about 0.4% by weight of phenoxyethanol as a preserving agent;

iv) about 0.05% by weight of docusate sodium (diethylhexyl sodium sulfosuccinate) as a surfactant;

v) about 4% by weight of glycerol as a humectant;

vi) about 0.2% by weight of poloxamer 124 as a liquid wetting surfactant;

vii) about 4% by weight of acrylamide, sodium acryloyldimethyl taurate copolymer, isohexadecane and polysorbate 80 as a gelling agent

viii) about 4% by weight of propylene glycol as a pro-penetrating agent;

ix) about 0.2% by weight of disodium EDTA as a sequestering agent; and

x) purified water as a vehicle.

In some embodiments, the gelling agent further comprises an emulsifying agent, such as sorbitan oleate. In some embodiments, the liquid wetting surfactant further comprises an antioxidant, such as tocopherol.

In some embodiments, the minimum amount of Isopropylcarbonate Benzoyl Peroxide remaining at the end of the shelf life of the composition is from about 0.001% to about 5%, about 0.001% to about 2%, about 0.001% to about 1.4%, about 0.5% to about 2%, or about 0.5% to about 1.4% by weight. In some embodiments, the minimum amount of Isopropylcarbonate Benzoyl Peroxide remaining at the end of the shelf life of the composition is from about 1.4% by weight.

Provided in another aspect is a method for producing an Isopropylcarbonate Benzoyl Peroxide aqueous gel, which method comprises the steps of:
i) solubilizing a sequestering agent in aqueous solution to produce the main phase;
ii) preparing a disaggregation medium comprising Isopropylcarbonate Benzoyl Peroxide by dispersing Isopropylcarbonate Benzoyl Peroxide, propylene glycol, glycerol and liquid wetting surfactant in water to produce medium A; and
iii) adding titanium dioxide to the main phase;
iv) adding a first portion of gelling agent to the main phase;
v) adding medium A to the main phase;
vi) adding of phenoxyethanol to the main phase;
vii) solubilizing docusate sodium in water to provide solubilized docusate sodium followed by addition of the solubilized docusate sodium to the main phase by gentle agitation; and
viii) adding a second portion of gelling agent to the main phase, whereby a gel is formed.

In some embodiments, the Isopropylcarbonate Benzoyl Peroxide from step ii) is subjected to a colloid mill.

Provided in another aspect is a method for treating common acne, comedones, polymorphous acne, nodulocystic acne, acne conglobata, or secondary acne afflicting the skin of an individual in need of such treatment, comprising topically administering to the individual any of the topically applicable compositions described herein.

Provided in another aspect is a regime or regimen for reducing the number of acne lesions, comprising administering to an individual afflicted therewith an effective amount of any one of the compositions described herein. In some embodiments, the acne lesions being of inflammatory and/or non-inflammatory type.

Provided in another aspect is a regimen for treatment of acne vulgaris, comprising,
i) cleaning skin;
ii) applying any one of the topically applicable compositions described herein to the skin; and
iii) applying a moisturizing treatment to the skin.

Provided in another aspect is a regimen for treatment of acne vulgaris, comprising,
i) cleaning skin; and
ii) applying any one of the topically applicable compositions described herein to the skin.

In some embodiments, the regimen for the treatment of acne vulgaris further comprises administering said topically applicable composition for at least twelve (12) months. In some embodiments, the regimen for the treatment of acne vulgaris further comprises administering said topically applicable composition for at least nine (9) months. In some embodiments, the regimen for the treatment of acne vulgaris further comprises administering said topically applicable composition once or twice a day. In some embodiments, the regimen for the treatment of acne vulgaris further comprises administering said topically applicable composition once a day. In some embodiments, the regimen for the treatment of acne vulgaris further comprises administering said topically applicable composition twice a day. In some embodiments, the regimen for the treatment of acne vulgaris further comprises administering said topically applicable composition every two (2) days. In some embodiments, the regimen for the treatment of acne vulgaris comprises administering said topically applicable composition in the evening after washing said affected skin area. In some embodiments, the said affected skin area contains from 20 to 100 non-inflammatory lesions, 20 to 50 inflammatory lesions, and no active nodules or cysts.

Provided in another aspect is a regime or regimen for controlling breakouts, comprising administering to an individual afflicted therewith an effective amount of any one of the compositions described herein. In some embodiments, controlling breakouts includes targeting at least one cause of blemishes and blackheads, including but not limited to purifying and cleansing the skin, un-clogging pores, reducing oil, and hydrating skin. In some embodiments, controlling breakouts includes targeting all four causes of blemishes.

Provided in another aspect is a kit comprising

a) any one of the topically applicable compositions described herein; and

b) a topical cleanser and/or a topical moisturizer.

In some embodiments, the facial cleaner and/or the facial moisturizer comprise salicylic acid. In some embodiments, the salicylic acid is present in an amount from 0.1-2.5%. In some embodiments, the salicylic acid is present in an amount of 0.5%. In some embodiments, the salicylic acid is present in an amount of 2%. In some embodiments, the facial cleaner and/or the facial moisturizer comprise benzoyl peroxide. In some embodiments, the benzoyl peroxide is present in an amount from 0.1-2.5%. In some embodiments, the benzoyl peroxide is present in an amount of 0.5%. In some embodiments, the benzoyl peroxide is present in an amount of 2%. In some embodiments, the facial cleaner and/or the facial moisturizer comprises adapalene. In some embodiments, the adapalene is present in an amount from 0.1-3%. In some embodiments, the adapalene is present in an amount of 1%.

### BRIEF DESCRIPTION OF DRAWINGS

FIGS. 1A and 1B show the change in subjects' blackheads according to the examples.

FIGS. 2A and 2B show the change in subjects' microcysts according to the examples.

FIGS. 3A and 3B show the change in subjects' non-inflammatory lesions according to the examples.

FIGS. 4A and 4B show the change in subjects' papules according to the examples.

FIGS. 5A and 5B show the change in subjects' inflammatory lesions according to the examples.

FIGS. 6A and 6B show the change in subjects' lesions according to the examples.

FIGS. 7A and 7B show the change in subjects' skin texture according to the examples.

FIGS. 8A and 8B show the change in subjects' skin uniformity according to the examples.

FIGS. 9A and 9B show the change in subjects' skin radiance according to the examples.

FIGS. 10A and 10B show the change in subjects' skin pore size according to the examples.

FIGS. 11A and 11B show the moisturizing effect to subjects' skin according to the examples.

FIGS. 12A and 12B show the sebo-regulating effect to subjects' skin according to the examples.

FIG. 13 shows a summary of the skin effects experienced by subjects according to the examples.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

### Definitions

As used herein, the singular forms “a,” “an,” and “the” designate both the singular and the plural, unless expressly stated to designate the singular only.

It is to be understood, although not always explicitly stated, that all numerical designations are preceded by the term “about.” The term “about” means that the number comprehended is not limited to the exact number set forth herein, and is intended to refer to numbers substantially around the recited number while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 5%, 1%, or 0.1% of the particular value.

Also as used herein, “and/or” refers to and encompasses any and all possible combinations of one or more of the associated listed items, as well as the lack of combinations when interpreted in the alternative (“or”).

The terms “administer,” “administration,” or “administering” as used herein refer to (1) providing, giving, dosing and/or prescribing, such as by either a health professional or his or her authorized agent or under his direction, and (2) putting into, taking or consuming, such as by a health professional or the subject. Administration shall include without limitation, administration by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. The invention is not limited by the route of administration, the formulation or dosing schedule.

The term “aqueous gel” means a composition containing, in an aqueous phase, a viscoelastic mass formed from colloidal suspensions (gelling agent).

“Comprising” shall mean that the methods and compositions include the recited elements, but not exclude others. “Consisting essentially of” when used to define methods and compositions, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transitional terms and phrases are within the scope of this invention.

A “therapeutically effective amount” in general means the amount that, when administered to a subject or animal for treating a disease, is sufficient to affect the desired degree of treatment for the disease. A “therapeutically effective amount” or a “therapeutically effective dosage” preferably treats or prevents any one of the diseases described herein, such as acne. Effective amounts of a compound or composition described herein thereof for treatment of a mammalian subject include, but are not limited to, about 0.1 to about 1000 mg/Kg of body weight of the subject/day, such as from about 1 to about 100 mg/Kg/day, especially from about 10 to about 100 mg/Kg/day. A broad range of disclosed composition dosages are believed to be both safe and effective.

The terms “treat”, “treating” or “treatment”, as used herein, include alleviating, abating or ameliorating any one of the diseases or disorders described herein, such as acne, one or more symptoms thereof, whether or not disease or disorder is considered to be “cured” or “healed” and whether or not all symptoms are resolved. The terms also include reducing or preventing progression of any one diseases or disorders described herein or one or more symptoms thereof, impeding or preventing an underlying mechanism of any one of the diseases or disorders described herein or one or more symptoms thereof, and achieving any therapeutic and/or prophylactic benefit.

As used herein, the term “subject” is used interchangeably with “patient,” and indicates a mammal, in particular a human, equine, bovine, porcine, feline, canine, murine, rat, or non-human primate. In preferred embodiments, the subject is a human.

### Compositions

Isopropylcarbonate Benzoyl Peroxide is an unstable peroxide derivative that releases benzoic acid and salicylic acid upon contact with skin. The potential for Isopropylcarbonate Benzoyl Peroxide in use for anti-acne treatment has been demonstrated, for instance in WO 2011/070170, which is incorporated by reference for the disclosure of this compound.

Formulating Isopropylcarbonate Benzoyl Peroxide into a composition or formulation suitable for dermatological or cosmetic use is challenging due to the chemical instability of Isopropylcarbonate Benzoyl Peroxide. Similar to benzoyl peroxide, the efficacy of Isopropylcarbonate Benzoyl Peroxide is associated with its decomposition when placed in contact with skin. The oxidizing properties of free radicals produced from this decomposition lead to the desired effect. Thus, in order to maintain the optimum efficacy of Isopropylcarbonate Benzoyl Peroxide, it is important to prevent its decomposition before use, i.e., during storage.

This disclosure provides compositions comprising Isopropylcarbonate Benzoyl Peroxide that have good physical, chemical and microbiological stability and corresponding methods of use. It is the present Inventors that recognized that the chemical degradation of Isopropylcarbonate Benzoyl Peroxide led to undesired discoloration of the compositions and bacterial growth also affects the stability of the composition. The use of an opacifier, such as titanium dioxide, masks the undesired discoloration; however, the use of an opacifier also destabilizes the compositions, which requires the use of specific components to counter the instability. Further, in some embodiments, a pro-penetrating agent, such as propylene glycol, and a liquid wetting surfactant, such as Poloxamer 124, maintain desired viscosity over time; however, the compositions are less chemically stable. In some embodiments, the Inventors learned that the addition of a sequestering agent, such as disodium EDTA, helps counter the chemical instability resulting from the use of a pro-penetrating agent and a liquid wetting surfactant. A preserving agent, such as phenoxyethanol, reduces and/or prevents undesired bacterial growth.

Described herein in one aspect is a topically applicable composition comprising:

i) Isopropylcarbonate Benzoyl Peroxide;

ii) an opacifier; and

iii) a preserving agent.

The compositions described herein also provide a minimum amount of Isopropylcarbonate Benzoyl Peroxide remaining at the end of the shelf life of the composition. In some embodiments, the minimum amount of Isopropylcarbonate Benzoyl Peroxide remaining at the end of the shelf life of the composition is from about 0.001% to about 5%, about 0.001% to about 2%, about 0.001% to about 1.4%, about 0.1% to about 5%, about 0.1% to about 2%, about 0.1% to about 1.4%, about 0.5% to about 2%, or about 0.5% to about 1.4% by weight. In some embodiments, the minimum amount of Isopropylcarbonate Benzoyl Peroxide remaining at the end of the shelf life of the composition is from about 0.001% to about 5%, about 0.001% to about 2%, about 0.001% to about 1.4%, about 0.5% to about 2%, or about 0.5% to about 1.4% by weight. In some embodiments, the minimum amount of Isopropylcarbonate Benzoyl Peroxide remaining at the end of the shelf life of the composition is about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2% by weight. In some embodiments, the minimum amount of Isopropylcarbonate Benzoyl Peroxide remaining at the end of the shelf life of the composition is about 1.4% by weight. In some embodiments, the shelf life of the composition is about 1 month, about 2 months, about 3 months, about 6 months, about 9 months, about 12 months, about 24 months, about 36 months, about 48 months, or about 60 months.

### Isopropylcarbonate Benzoyl Peroxide

As used herein, Isopropylcarbonate Benzoyl Peroxide has the following structure:

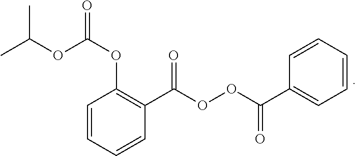 [SMILES: CC(C)OC(=O)Oc1ccccc1C(=O)OOC(=O)c1ccccc1]

Isopropylcarbonate Benzoyl Peroxide has a molecular formula of C₁₈H₁₆O₇ and molecular weight of 344.32 g/mol. The CAS number is 1310672-91-3. Isopropylcarbonate Benzoyl Peroxide is described in WO 2011/070170, which is incorporated by reference for the disclosure of this compound.

In some embodiments, the composition comprises about 0.0001% to about 20% by weight of Isopropylcarbonate Benzoyl Peroxide. In some embodiments, the composition comprises about 0.001% to about 20% by weight of Isopropylcarbonate Benzoyl Peroxide. In some embodiments, the composition comprises about 0.01% to about 20% by weight of Isopropylcarbonate Benzoyl Peroxide. In some embodiments, the composition comprises about 0.1% to about 20% by weight of Isopropylcarbonate Benzoyl Peroxide. In some embodiments, the composition comprises about 0.1% to about 10% by weight of Isopropylcarbonate Benzoyl Peroxide. In some embodiments, the composition comprises about 0.1% to about 5% by weight of Isopropylcarbonate Benzoyl Peroxide. In some embodiments, the composition comprises about 2.5% to about 10% by weight of Isopropylcarbonate Benzoyl Peroxide. In some embodiments, the composition comprises about 2.5% to about 5% by weight of Isopropylcarbonate Benzoyl Peroxide.

In some embodiments, the composition comprises about 0.0001%, about 0.001%, about 0.01%, about 0.1%, about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% by weight of Isopropylcarbonate Benzoyl Peroxide. In some embodiments, the composition comprises about 2.5% by weight of Isopropylcarbonate Benzoyl Peroxide. In some embodiments, the composition comprises about 3% by weight of Isopropylcarbonate Benzoyl Peroxide. In some embodiments, the composition comprises about 5% by weight of Isopropylcarbonate Benzoyl Peroxide. In some embodiments, the composition comprises about 10% by weight of Isopropylcarbonate Benzoyl Peroxide.

### Opacifiers

As used herein, an opacifier is an agent that is added to a formulation in order to make the formulation opaque. In some embodiments, the opacifier prevents the discoloration of the formulation that is observed over time, which may be a result of the degradation of the active agent. Examples of suitable opacifiers include, but are not limited to, bismuth oxychloride, titanium dioxide, fluorphlogopite, mica, iron oxide, nylon polymer, polymethyl methacrylate, boron nitride, kaolin, glycol distearate, styrene copolymer, a fatty alcohol, and any combination thereof. Other examples include, but are not limited to, PERLITE® 02uvs (bismuth oxychloride); SunSHINE® soft white (TiO2+fluorphlogopite); COLORONA® imperial citrine (mica/iron oxide); TIMIRON® super sheen (mica/TiO2); ORGASOL® 2002 EXT (Nylon 12); SunPMMA-S (PMMA, polymethyl methacrylate); Orange k7001-j, RONAFLAIR® BORONEIGE® SPF3 (boron nitride; and WATER BN™ 3002 (Boron Nitride/PEG-8 methyl ether dimethicone). In some embodiments, the opacifier is bismuth oxychloride, titanium dioxide, fluorphlogopite, mica, iron oxide, nylon polymer, polymethyl methacrylate, boron nitride, kaolin, glycol distearate, styrene copolymer, a fatty alcohol, and any combination thereof. In some embodiments, the opacifier is bismuth oxychloride, titanium dioxide, fluorphlogopite, mica, iron oxide, nylon polymer, polymethyl methacrylate, boron nitride, and any combination thereof. In some embodiments, the opacifier is titanium dioxide. The opacifier is preferably pharmaceutical grade or cosmetic grade. In some embodiments, the opacifier, such as titanium dioxide, is pharmaceutical grade. In some embodiments, the opacifier, such as titanium dioxide, is cosmetic grade.

In some embodiments, the composition comprises about 0.1% to about 10% by weight of the opacifier. In some embodiments, the composition comprises about 0.1% to about 5% by weight of the opacifier. In some embodiments, the composition comprises about 0.1% to about 2.5% by weight of the opacifier. In some embodiments, the composition comprises about 0.1% to about 1% by weight of the opacifier. In some embodiments, the composition comprises about 0.1% to about 0.5% by weight of the opacifier. In some embodiments, the composition comprises about 0.2% to about 2.5% by weight of the opacifier. In some embodiments, the composition comprises about 0.4% by weight of the opacifier.

In some embodiments, the composition comprises about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight of the opacifier. In some embodiments, the composition comprises about 0.2% by weight of the opacifier. In some embodiments, the composition comprises about 0.4% by weight of the opacifier. In some embodiments, the composition comprises about 1% by weight of the opacifier. In some embodiments, the composition comprises about 2.5% by weight of the opacifier.

### Preserving Agents

As used herein, an preserving agent is used in the formulation to reduce or prevent growth from bacteria, such as Burkholderia cepacia. The growth of such bacteria, in some embodiments, affects the stability of the compositions described herein. Examples of suitable preserving agents include, but are not limited to, phenoxyethanol, potassium sorbate, benzyl alcohol, methyl paraben (NIPAGIN® M), chlorhexidine digluconate, chloroxylenol, chlorphenesin, dehydroacetic acid, diazolidinyl urea, DMDM hydantoin, ethylparaben, iodopropynyl butylcarbamate, methylisothiazolinone, propyllparaben, phenoxyethanol, phenoxyisopropanol, polyaminopropyl biguianide, sodium benzoate, salicylic acid, and any combination thereof. In some embodiments, the preserving agent is phenoxyethanol, potassium sorbate, benzyl alcohol, methyl paraben, chlorhexidine digluconate, chloroxylenol, chlorphenesin, dehydroacetic acid, diazolidinyl urea, DMDM hydantoin, ethylparaben, iodopropynyl butylcarbamate, methylisothiazolinone, propyllparaben, phenoxyethanol, phenoxyisopropanol, polyaminopropyl biguianide, sodium benzoate, salicylic acid, or any combination thereof. In some embodiments, the preserving agent is phenoxyethanol, potassium sorbate, benzyl alcohol, methyl paraben, or any combination thereof. In some embodiments, the preserving agent is phenoxyethanol.

In some embodiments, the composition comprises about 0.1% to about 10% by weight of the preserving agent. In some embodiments, the composition comprises about 0.1% to about 5% by weight of the preserving agent. In some embodiments, the composition comprises about 0.1% to about 2.5% by weight of the preserving agent. In some embodiments, the composition comprises about 0.1% to about 1% by weight of the preserving agent. In some embodiments, the composition comprises about 0.1% to about 0.8% by weight of the preserving agent.

In some embodiments, the composition comprises about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight of the preserving agent. In some embodiments, the composition comprises about 0.1% by weight of the preserving agent. In some embodiments, the composition comprises about 0.2% by weight of the preserving agent. In some embodiments, the composition comprises about 0.4% by weight of the preserving agent. In some embodiments, the composition comprises about 0.5% by weight of the preserving agent. In some embodiments, the composition comprises about 0.8% by weight of the preserving agent.

### Surfactants

In some embodiments, the compositions described herein further comprise a surfactant. Examples of suitable surfactants include, but are not limited to, docusate sodium (diethylhexyl sodium sulfosuccinate), poloxamers, PEG ethers, PPG esters, alkyl polyglucosides, sorbitan esters, ethoxylated sorbitan esters, alcohol sulfates, ethoxylated alcohol sulfates, alkyl benzene sulfonates, alpha olefin sulfonates, sulfosuccinates, isethionate esters, taurates, alkyl betaines, alkyl amidopropyl betaines, amphoacetates, or alkyl sultaines. In some embodiments, the composition further comprises a surfactant. In some embodiments, the surfactant is docusate sodium, diethyl sodium sulfosuccinate, poloxamer, PEG ether, PPG ester, alkyl polyglucoside, sorbitan ester, ethoxylated sorbitan ester, alcohol sulfate, ethoxylated alcohol sulfate, alkyl benzene sulfonate, alpha olefin sulfonate, sulfosuccinate, isethionate ester, taurate, alkyl betaine, alkyl amidopropyl betaine, amphoacetate, or alkyl sultaine. In some embodiments, the surfactant is a sulfosuccinate. In some embodiments, the surfactant is docusate sodium (diethylhexyl sodium sulfosuccinate or also known as dioctyl sodium sulfosuccinate. In some embodiments, the surfactant is diethylhexyl sodium sulfosuccinate.

In some embodiments, the composition comprises about 0.01% to about 10% by weight of the surfactant. In some embodiments, the composition comprises about 0.01% to about 5% by weight of the surfactant. In some embodiments, the composition comprises about 0.01% to about 2.5% by weight of the surfactant. In some embodiments, the composition comprises about 0.01% to about 1% by weight of the surfactant. In some embodiments, the composition comprises about 0.01% to about 0.1% by weight of the surfactant. In some embodiments, the composition comprises about 0.05% by weight of the surfactant.

In some embodiments, the composition comprises about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight of the surfactant. In some embodiments, the composition comprises about 0.01% by weight of the surfactant. In some embodiments, the composition comprises about 0.05% by weight of the surfactant. In some embodiments, the composition comprises about 0.1% by weight of the surfactant. In some embodiments, the composition comprises about 0.2% by weight of the surfactant.

### Humectants

In some embodiments, the compositions described herein further comprise a humectant. Examples of suitable humectants include, but are not limited to, glycerol and sorbitol. In some embodiments, the humectant is glycerol.

In some embodiments, the composition comprises about 0.1% to about 20% by weight of the humectant. In some embodiments, the composition comprises about 0.1% to about 10% by weight of the humectant. In some embodiments, the composition comprises about 0.1% to about 5% by weight of the humectant. In some embodiments, the composition comprises about 0.1% to about 2.5% by weight of the humectant. In some embodiments, the composition comprises about 0.1% to about 1% by weight of the humectant. In some embodiments, the composition comprises about 1% to about 10% by weight of the humectant. In some embodiments, the composition comprises about 4% by weight of the humectant.

In some embodiments, the composition comprises about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% by weight of the humectant. In some embodiments, the composition comprises about 1% by weight of the humectant. In some embodiments, the composition comprises about 2% by weight of the humectant. In some embodiments, the composition comprises about 4% by weight of the humectant. In some embodiments, the composition comprises about 5% by weight of the humectant. In some embodiments, the composition comprises about 10% by weight of the humectant.

### Liquid Wetting Surfactants

In some embodiments, the compositions described herein further comprise a liquid wetting surfactant. The wetting power is the tendency of a liquid to spread over a surface. Suitable liquid wetting surfactants are preferably surfactants with an HLB (Hydrophilic-Lipophilic Balance) value from 7 to 9, or nonionic surfactants such as polyoxyethylenated and/or polyoxypropylenated copolymers. In some embodiments, such liquid wetting surfactants are liquid so as to be readily incorporated into the composition without it being necessary to heat them. Among the liquid wetting surfactants that are preferably used, without this list being limiting, are compounds of the poloxamer family and more particularly poloxamer 124 and/or poloxamer 182. In some embodiments, the liquid wetting surfactant is poloxamer. In some embodiments, the liquid wetting surfactant is poloxamer 124. In some embodiments, the liquid wetting surfactant, such as a poloxamer, further comprises an antioxidant, such as tocopherol.

In some embodiments, the composition comprises about 0.01% to about 10% by weight of the liquid wetting surfactant. In some embodiments, the composition comprises about 0.01% to about 5% by weight of the liquid wetting surfactant. In some embodiments, the composition comprises about 0.01% to about 2.5% by weight of the liquid wetting surfactant. In some embodiments, the composition comprises about 0.01% to about 1% by weight of the liquid wetting surfactant. In some embodiments, the composition comprises about 0.01% to about 0.5% by weight of the liquid wetting surfactant. In some embodiments, the composition comprises about 0.1% to about 5% by weight of the liquid wetting surfactant. In some embodiments, the composition comprises about 0.1% to about 2% by weight of the liquid wetting surfactant.

In some embodiments, the composition comprises about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight of the liquid wetting surfactant. In some embodiments, the composition comprises about 0.1% by weight of the liquid wetting surfactant. In some embodiments, the composition comprises about 0.2% by weight of the liquid wetting surfactant. In some embodiments, the composition comprises about 0.3% by weight of the liquid wetting surfactant. In some embodiments, the composition comprises about 0.4% by weight of the liquid wetting surfactant. In some embodiments, the composition comprises about 0.5% by weight of the liquid wetting surfactant.

### Gelling Agents

In some embodiments, the composition further comprises a gelling agent. Suitable examples of gelling agents include, but are not limited to, acrylates/Steareth-20 methacrylate copolymer, acrylamide/sodium acrylate copolymer, acrylamide/sodium acryloyl dimethyltaurate copolymer/isohexadecane/polysorbate-20, acrylamide/ammonium acrylate copolymer, polyisobutene, polysorbate 20, acrylamidopropyltrimonium chloride/acrylamide copolymer, acrylates copolymer, acrylates/C10-30 alkyl acrylates crosspolymer, acrylates/acrylamide copolymer and mineral oil and polysorbate-85, acrylates/C12-22 alkyl methacrylate copolymer, acrylates/vinyl ssodecanoate croospolymer, acrylic acid copolymer, ammomium acryloyldimethyltaurate/beheneth-25 methacrylate copolymer, ammomium acryloyldimethyltaurate/VP copolymer, ammonium polyacrylate/isohexadecane/PEG 40 castor oil/sorbitan oleate/aqua, biosaccharide gum-1, bentonite/xanthan gum (smectite), C13-14 isoparafin/mineral oil/sodium polyacrylate/polyacrylamide/polysorbate 85 carbomer, carboxyvinyl polymer, cellulose gum, glyceryl polymethacrylate, hydrogenated polydecene, ethylene/propylene/styrene copolymer, butylene/ethylene/styrene copolymer, hydroxyethyl acrylate/sodium acryloyldimethyltaurate copolymer/squalane/polysorbate 60/water, hydroxyethylcellulose, hydroxypropyl starch phosphate, hydroxypropylcelulose, magnesium aluminium silicate, magnesium silicate, methyl vinyl ether/maleic anhydride (PVM/MA decadiene crosspolymer)), polyacrylamide/C13-14 Isoparafin/Laureth-7/aqua, polyacrylate 13/polyisobutene/polysorbate 20, potato starch modified, propane-1,2-diol alginate, PVP (polyvinylpyrrolidone), sclerotium gum, sodium acrylate copolymer/PPG-1 trideceth 6/parafinum liquidum/sorbitan trioleate/aqua, sodium acrylate/acryloyldimethyltaurate/copolymer & isohexadecane & polysorbate, sodium acrylate/acryloyldimethyltaurate, copolymer/polyisobutene/caprilyl capryl glucoside, sodium acrylates copolymer/glycine soja/PPG-1 trideceth-6 (anionic), sodium acrylates copolymer/parafinium liquidum/PPG-1 trideceth-6 (anionic), sodium acrylates copolymer/hydrogenated polyisobutene/phospholipids/polyglyceryl-10 stearate/Helianthus annuus seed oil, sodium carbomer, sodium polyacrylate, sodium polyacrylate/hydrogenated polydecane, steareth-10 allyl ether/acrylates copolymer, succinoglycan, water/glycerin/polyacrylimidomethylpropane/sulfonate/polyquaternium-4, xanthan gum, xanthan gum/magnesium aluminium silicate, xanthan gum/hectorite/cellulose, and magnesium aluminium silicate. Other examples of suitable gelling agents include, but are not limited, such as the mixture of acrylamide/sodium acryloyldimethyltaurate copolymer/isohexadecane/polysorbate 80 sold under the name SIMULGEL™ 600 by the company SEPPIC, the mixture of polyacrylamide/isoparaffin C13-14/laureth-7 such as, for example, the product sold under the name SEPIGEL305™ by the company SEPPIC, the family of acrylic polymers coupled to hydrophobic chains, such as the PEG-150/decyl/SMDI copolymer sold under the name ACULYN™ 44 (polycondensate comprising at least, as components, a polyethylene glycol containing 150 or 180 mol of ethylene oxide, decyl alcohol and methylenebis (4-cyclohexyl isocyanate) (SMDI), at 35% by weight in a mixture of propylene glycol (39%) and water (26%)), the family of modified starches, such as the modified potato starch sold under the name STRUCTURE® Solanace, or mixtures thereof. Other examples include the mixture of hydroxyethyl acrylate/sodium acryloyldimethyl taurate copolymer/isohexadecane/polysorbate 60 sold as SIMULGEL™ INS 100; the mixture of hydroxyethyl acrylate/sodium acryloyldimethyl taurate copolymer sold as SEPINOV™ EMT10; the mixture of polyacrylate-13/polyisobutene/polysorbate 20 sold as SEPIPLUS™ 400; the mixture of hydroxyethyl acrylate/sodium acryloyldimethyl taurate copolymer/polyisobutene/PEG-7 sold as SEPIPLUS™ S; the mixture sold as polyacrylate crosspolymer-6 sold as SEPIMAX ZEN™. In some embodiments, the gelling agent comprises an acrylamide, polyacrylamide, or acrylate. In some embodiments, the suitable gelling agents are cosmetic grade or pharmaceutical grade. In some embodiments, the gelling agent is cosmetic grade. In some embodiments, the gelling agent is pharmaceutical grade.

In some embodiments, the gelling agent comprises an acrylamide or is derived from the acrylamide family. In some embodiments, the gelling agent comprises acrylamide, sodium acryloyldimethyl taurate copolymer, isohexadecane and polysorbate 80 (SIMULGEL™ 600). In some embodiments, the gelling agent comprises acrylamide, sodium acryloyldimethyl taurate copolymer, isohexadecane, polysorbate 80, and optionally sorbitan oleate (SIMULGEL™ 600). In some embodiments, the gelling agent comprises an acrylate or is derived from the acrylate family. In some embodiments, the gelling agent comprises hydroxyethyl acrylate, sodium acryloyldimethyl taurate copolymer, and isohexadecane and polysorbate 60 (SIMULGEL™ INS 100). In some embodiments, the gelling agent comprises hydroxyethyl acrylate and sodium acryloyldimethyl taurate copolymer (SEPINOV™ EMT10).

In some embodiments, the composition comprises about 0.1% to about 20% by weight of the gelling agent. In some embodiments, the composition comprises about 0.1% to about 10% by weight of the gelling agent. In some embodiments, the composition comprises about 0.1% to about 5% by weight of the gelling agent. In some embodiments, the composition comprises about 0.1% to about 2.5% by weight of the gelling agent. In some embodiments, the composition comprises about 0.1% to about 1% by weight of the gelling agent. In some embodiments, the composition comprises about 1% to about 10% by weight of the gelling agent. In some embodiments, the composition comprises about 4% by weight of the gelling agent.

In some embodiments, the composition comprises about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% by weight of the gelling agent. In some embodiments, the composition comprises about 1% by weight of the gelling agent. In some embodiments, the composition comprises about 2% by weight of the gelling agent. In some embodiments, the composition comprises about 4% by weight of the gelling agent. In some embodiments, the composition comprises about 5% by weight of the gelling agent. In some embodiments, the composition comprises about 10% by weight of the gelling agent.

### Pro-Penetrating Agents

In some embodiments, the compositions described herein further comprise a pro-penetrating agents. Examples of suitable pro-penetrating agents include, but are not limited to, propylene glycol, dipropylene glycol, propylene glycol dipelargonate, lauroglycol, alcohols, alkylmethyl sulfoxides, polyols, oleic acid, isopropyl myristate, decylmethyl sulfoxide, DMSO, urea, and ethoxydiglycol. In some embodiments, the composition further comprises a pro-penetrating agent. In some embodiments, the pro-penetrating agent is propylene glycol, dipropylene glycol, propylene glycol dipelargonate, lauroglycol, an alcohol, an alkylmethyl sulfoxide, a polyol, oleic acid, isopropyl myristate, decylmethyl sulfoxide, DMSO, urea, or ethoxydiglycol. In some embodiments, the pro-penetrating agent is propylene glycol, dipropylene glycol, propylene glycol dipelargonate, lauroglycol, or ethoxydiglycol. In some embodiments, the pro-penetrating agent is propylene glycol.

In some embodiments, the composition comprises about 0.1% to about 20% by weight of the pro-penetrating agent. In some embodiments, the composition comprises about 0.1% to about 10% by weight of the pro-penetrating agent. In some embodiments, the composition comprises about 0.1% to about 5% by weight of the pro-penetrating agent. In some embodiments, the composition comprises about 0.1% to about 2.5% by weight of the pro-penetrating agent. In some embodiments, the composition comprises about 0.1% to about 1% by weight of the pro-penetrating agent. In some embodiments, the composition comprises about 1% to about 10% by weight of the pro-penetrating agent. In some embodiments, the composition comprises about 2% to about 6% by weight of the pro-penetrating agent. In some embodiments, the composition comprises about 4% by weight of the pro-penetrating agent.

In some embodiments, the composition comprises about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% by weight of the pro-penetrating agent. In some embodiments, the composition comprises about 1% by weight of the pro-penetrating agent. In some embodiments, the composition comprises about 2% by weight of the pro-penetrating agent. In some embodiments, the composition comprises about 4% by weight of the pro-penetrating agent. In some embodiments, the composition comprises about 5% by weight of the pro-penetrating agent. In some embodiments, the composition comprises about 10% by weight of the pro-penetrating agent.

### Sequestering Agents

In some embodiments, the compositions described herein further comprise a sequestering agent. Examples of suitable sequestering agents include, but are not limited to, disodium ethylenediaminetetraacetic acid (EDTA), dihydroxyethylglycine, trisodium ethylenediamine fisuccinate glutamic acid diacetic acid tetra sodium salt (GLDA), fiethylenetriaminepentaacetic acid (DTPA), methylglycindiacetic acid (MGDA), hydroxyethylethylenediaminetriacetic acid (HEDTA), glucoheptonate, nitrilotriacetic acid (NTA), ethylenediaminedisuccinic acid (EDDS), iminodisuccinic acid (IDS), ethylenediamine-N,N′-diglutaric acid (EDDG), ethylenediamine-N,N′-dimalonic acid (EDDM), 3-hydroxy-2,2-iminodisuccinic acid (HIDS), 2-hydroxyethyliminodiacetic acid (HEIDA), pyridine-2,6-dicarboxylic acid (PDA), etidronic acid. carnosine, phytic acid, kojic acid, sodium carboxymethyl inulin, citric acid, tartaric acid or a derivative or salt thereof. In some embodiments, the sequestering agent is disodium ethylenediaminetetraacetic acid (EDTA), dihydroxyethylglycine, trisodium ethylenediamine fisuccinate glutamic acid diacetic acid tetra sodium salt (GLDA), fiethylenetriaminepentaacetic acid (DTPA), methylglycindiacetic acid (MGDA), hydroxyethylethylenediaminetriacetic acid (HEDTA), glucoheptonate, nitrilotriacetic acid (NTA), ethylenediaminedisuccinic acid (EDDS), iminodisuccinic acid (IDS), ethylenediamine-N,N′-diglutaric acid (EDDG), ethylenediamine-N,N′-dimalonic acid (EDDM), 3-hydroxy-2,2-iminodisuccinic acid (HIDS), 2-hydroxyethyliminodiacetic acid (HEIDA), pyridine-2,6-dicarboxylic acid (PDA), etidronic acid. carnosine, phytic acid, kojic acid, sodium carboxymethyl inulin, citric acid, tartaric acid or a derivative or salt thereof. In some embodiments, the sequestering agent is disodium ethylenediaminetetraacetic acid (EDTA), dihydroxyethylglycine, citric acid, tartaric acid or a derivative or salt thereof. In some embodiments, the sequestering agent is disodium ethylenediaminetetraacetic acid (EDTA).

In some embodiments, the composition comprises about 0.01% to about 10% by weight of the sequestering agent. In some embodiments, the composition comprises about 0.01% to about 5% by weight of the sequestering agent. In some embodiments, the composition comprises about 0.01% to about 2.5% by weight of the sequestering agent. In some embodiments, the composition comprises about 0.01% to about 1% by weight of the sequestering agent. In some embodiments, the composition comprises about 0.01% to about 0.5% by weight of the sequestering agent. In some embodiments, the composition comprises about 0.1% to about 0.5% by weight of the sequestering agent. In some embodiments, the composition comprises about 0.1% to about 0.2% by weight of the sequestering agent.

In some embodiments, the composition comprises about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight of the sequestering agent. In some embodiments, the composition comprises about 0.01% by weight of the sequestering agent. In some embodiments, the composition comprises about 0.05% by weight of the sequestering agent. In some embodiments, the composition comprises about 0.1% by weight of the sequestering agent. In some embodiments, the composition comprises about 0.2% by weight of the sequestering agent. In some embodiments, the composition comprises about 0.3% by weight of the sequestering agent. In some embodiments, the composition comprises about 0.4% by weight of the sequestering agent. In some embodiments, the composition comprises about 0.5% by weight of the sequestering agent.

### Vehicles

In some embodiments, the compositions described herein further comprise a vehicle. Examples of suitable vehicles include, but are not limited to, water, a floral water such as cornflower water, or natural mineral or spring water chosen, for example, from eau de Vittel, waters of the Vichy basin, eau d'Uriage, eau de la Roche Posay, eau de la Bourboule, eau d'Enghien-les-Bains, eau de Saint Gervais-les-Bains, eau de Néris-les-Bains, eau d'Allevard-les-Bains, eau de Digne, eau de Maizières, eau de Neyrac-les-Bains, eau de Lons-le-Saunier, les Eaux Bonnes, eau de Rochefort, eau de Saint Christau, eau des Fumades, eau de Tercis-les-bains, eau d'Avène or eau d'Aix les Bains. In some embodiments, the vehicle is purified water.

In some embodiments, the composition comprises about 10% to about 90% or about 20% to about 80% by weight of the vehicle.

Provided in one aspect is a topically applicable composition comprising:

i) Isopropylcarbonate Benzoyl Peroxide;

ii) an opacifier;

iii) a preserving agent;

iv) a surfactant;

v) a humectant;

vi) a liquid wetting surfactant;

vii) a gelling agent;

viii) a pro-penetrating agent;

ix) a sequestering agent; and

x) purified water as a vehicle.

Provided in one aspect is a topically applicable composition comprising:

i) about 0.0001% to about 20% by weight of Isopropylcarbonate Benzoyl Peroxide;

ii) about 0.1% to about 10% by weight of an opacifier;

iii) about 0.1% to about 10% by weight of a preserving agent;

iv) about 0.01% to about 10% by weight of a surfactant;

v) about 0.1% to about 20% by weight of a humectant;

vi) about 0.01% to about 10% by weight of a liquid wetting surfactant;

vii) about 0.1% to about 20% by weight of a gelling agent;

viii) about 0.1% to about 20% by weight of a pro-penetrating agent;

ix) about 0.01% to about 10% by weight of a sequestering agent; and

x) purified water as a vehicle.

Provided in one aspect is a topically applicable composition comprising:

i) Isopropylcarbonate Benzoyl Peroxide;

ii) titanium dioxide as an opacifier;

iii) phenoxyethanol as a preserving agent;

iv) docusate sodium (diethylhexyl sodium sulfosuccinate) as a surfactant;

v) glycerol as a humectant;

vi) poloxamer 124 as a liquid wetting surfactant;

vii) a combination of acrylamide, sodium acryloyldimethyl taurate copolymer, isohexadecane, and polysorbate 80 as a gelling agent;

viii) propylene glycol as a pro-penetrating agent;

ix) disodium EDTA as a sequestering agent; and

x) purified water as a vehicle.

Provided in one aspect is a topically applicable composition comprising:

i) about 0.1% to about 10% by weight of Isopropylcarbonate Benzoyl Peroxide;

ii) about 0.1% to about 5% by weight of titanium dioxide as an opacifier;

iii) about 0.1% to about 2.5% by weight of phenoxyethanol as a preserving agent;

iv) about 0.01% to about 2.5% by weight of docusate sodium (diethylhexyl sodium sulfosuccinate) as a surfactant;

v) about 0.1% to about 10% by weight of glycerol as a humectant;

vi) about 0.1% to about 5% by weight of poloxamer 124 as a liquid wetting surfactant;

vii) about 0.1% to about 10% by weight of a combination of acrylamide, sodium acryloyldimethyl taurate copolymer, isohexadecane, and polysorbate 80 as a gelling agent

viii) about 0.1% to about 10% by weight of propylene glycol as a pro-penetrating agent;

ix) about 0.1% to about 10% by weight of disodium EDTA as a sequestering agent; and

x) purified water as a vehicle.

Provided in one aspect is a topically applicable composition comprising:

i) about 3% by weight of Isopropylcarbonate Benzoyl Peroxide;

ii) about 0.4% by weight of titanium dioxide as an opacifier;

iii) about 0.4% by weight of phenoxyethanol as a preserving agent;

iv) about 0.05% by weight of docusate sodium (diethylhexyl sodium sulfosuccinate) as a surfactant;

v) about 4% by weight of glycerol as a humectant;

vi) about 0.2% by weight of poloxamer 124 as a liquid wetting surfactant;

vii) about 4% by weight of a combination of acrylamide, sodium acryloyldimethyl taurate copolymer, isohexadecane, and polysorbate 80 as a gelling agent

viii) about 4% by weight of propylene glycol as a pro-penetrating agent;

ix) about 0.1% or about 0.2% by weight of disodium EDTA as a sequestering agent; and

x) purified water as a vehicle.

Provided in one aspect is a topically applicable composition comprising:

i) about 3% by weight of Isopropylcarbonate Benzoyl Peroxide;

ii) about 0.4% by weight of titanium dioxide as an opacifier;

iii) about 0.4% by weight of phenoxyethanol as a preserving agent;

iv) about 0.05% by weight of docusate sodium (diethylhexyl sodium sulfosuccinate) as a surfactant;

v) about 4% by weight of glycerol as a humectant;

vi) about 0.2% by weight of poloxamer 124 as a liquid wetting surfactant;

vii) about 4% by weight of a combination of acrylamide, sodium acryloyldimethyl taurate copolymer, isohexadecane, and polysorbate 80 as a gelling agent

viii) about 4% by weight of propylene glycol as a pro-penetrating agent;

ix) about 0.2% by weight of disodium EDTA as a sequestering agent; and

x) purified water as a vehicle.

In some embodiments, the gelling agent further comprises an emulsifying agent, such as sorbitan oleate. In some embodiments, the liquid wetting surfactant further comprises an antioxidant, such as tocopherol.

### Additional Components

In some embodiments, the composition described herein further comprises a keratolytic agent, an oil, an antioxidant, a skin conditioning agent, or any combination thereof. In some embodiments, the composition further comprises an antioxidant.

In some embodiments, the keratolytic agent is lactic acid, glycolic acid, malic acid, phytic acid, silver or a silver solution. In some embodiments, the composition comprises about 0.01% to about 10%, 0.01% to about 5%, or 0.01% to about 2.5% by weight of the keratolytic agent. In some embodiments, the composition comprises about 0.05% or about 2% by weight of the keratolytic agent.

In some embodiments, the oil comprises mineral oil, hydrogenated polyisobutene, squalene, polydecene, or any combination thereof. In some embodiments, the oil is hydrogenated polyisobutene. In some embodiments, the oil is squalene. In some embodiments, the oil is a combination of hydrogenated polyisobutene and squalene. In some embodiments, the composition comprises about 0.1% to about 20%, about 0.1% to about 10%, 0.1% to about 5%, or 0.1% to about 2.5% by weight of the oil. In some embodiments, the composition comprises about 10% by weight of the oil.

In some embodiments, the antioxidant comprises butylated hydroxytoluene, DL alpha tocopherol, ascorbyl palmitate, or any combination thereof. In some embodiments, the antioxidant is butylated hydroxytoluene. In some embodiments, the antioxidant is DL alpha tocopherol. In some embodiments, the antioxidant is ascorbyl palmitate. In some embodiments, the antioxidant is a combination of DL alpha tocopherol and ascorbyl palmitate. In some embodiments, the composition comprises about 0.001% to about 10%, about 0.001% to about 1%, 0.01% to about 1%, or 0.1% to about 1% by weight of the antioxidant. In some embodiments, the composition comprises about 0.1% or about 0.025% by weight of the antioxidant.

In some embodiments, the skin conditioning agent comprises silica beads, methyl methacrylate cross polymer, nylon polymer, mica, polymethyl methacrylate, titanium dioxide, or any combination thereof. Other examples include, but not limited to, Sunsil 150-H (Silica beads); Sun PMMA-S (methyl methacrylate cross polymer); Sun PMMA-X (methyl methacrylate cross polymer); ORGASOL® 2002 EXD Nat (Nylon12); TIMIRON® Super Sheen MP-1001 (Mica 64%, TiO2 45%); and JH-Gold (mica, polymethyl methacrylate, titanium dioxide). In some embodiments, the composition comprises about 0.1% to about 20%, about 0.1% to about 10%, 0.1% to about 5%, or 0.1% to about 2% by weight of the skin conditioning agent. In some embodiments, the composition comprises about 0.1%, about 0.5%, about 0.7%, about 1%, about 2%, about 3%, or about 5% by weight of the skin conditioning agent.

In some embodiments, the composition may also comprise any additive usually used in the cosmetics or pharmaceutical field, such as sequestering agents, antioxidants, sunscreens, preserving agents, fillers, electrolytes, humectants, colorants, common mineral or organic acids or bases, fragrances, essential oils, cosmetic active agents, moisturizers, vitamins, essential fatty acids, sphingolipids, self-tanning compounds such as DHA, and calmants and protective agents for the skin such as allantoin. Needless to say, a person skilled in the art will take care to select this or these optional additional compound(s), and/or the amount thereof, such that the advantageous properties of the composition according to the invention are not, or are not substantially, adversely affected.

### Stability

Stability as referenced herein refers to at the predetermined time limit, that the composition comprises less than about 10%, less than about 5%, less than about 2.5%, or less than about 1% by weight of degradation products or products. Or alternatively, stability refers to at the predetermined time limit that the composition comprises greater than about 60%, greater than about 70%, greater than about 80%, greater than about 90%, greater than about 95%, greater than about 97.5%, or greater than 99% by weight of Isopropylcarbonate Benzoyl Peroxide. In some embodiments, the compositions described herein are stable. Preferentially, the compositions are stable for at least 1 month, at least 2 months, at least 3 months, at least 6 months, at least 9 months, at least 12 months, at least 24 months, or at least 36 months at about 5° C., about 25° C., about 30° C., or about 40° C. In some embodiments, the compositions are stable for about 1 month, about 2 months, about 3 months, about 6 months, about 9 months, about 12 months, about 24 months, or about 36 months at about 5° C., about 25° C., about 30° C., or about 40° C.

In some embodiments, the compositions described herein are stable. In some embodiments, the compositions are stable for at least 1 month, at least 2 months, at least 3 months, at least 6 months, at least 9 months, at least 12 months, at least 24 months, or at least 36 months at about 25° C. and 60% relative humidity. In some embodiments, the compositions are stable for about 1 month, about 2 months, about 3 months, about 6 months, about 9 months, about 12 months, about 24 months, or about 36 months at about 25° C. and 60% relative humidity.

In some embodiments, the compositions described herein are stable. In some embodiments, the compositions are stable for at least 1 month, at least 2 months, at least 3 months, at least 6 months, at least 9 months, at least 12 months, at least 24 months, or at least 36 months at about 30° C. or 40° C. and 75% relative humidity. In some embodiments, the compositions are stable for about 1 month, about 2 months, about 3 months, about 6 months, about 9 months, about 12 months, about 24 months, or about 36 months at about 30° C. or 40° C. and 75% relative humidity.

### Formulations

In some embodiments, the compositions described herein are formulated as gels. In some embodiments, the compositions described herein are formulated as creams. In some embodiments, the compositions described herein a suitable for topical administration.

### Methods and Regimens

Provided in one aspect is a method for treating common acne, comedones, polymorphous acne, nodulocystic acne, acne conglobata, or secondary acne afflicting the skin of an individual in need of such treatment, comprising topically administering to the individual any one of the topically applicable compositions described herein.

In some embodiments, the topically applicable composition is administered daily, every other day, twice per week, three times per week, four times per week, five times per week, six times per week, once per week, once every two weeks, once every three weeks, once every four weeks, once every five weeks, once every six weeks, once every seven weeks, once every eight weeks, once every nine weeks, once every 10 weeks, once every 11 weeks, once every 12 weeks, twice per year, once per year, and/or as needed based on the appearance of symptoms of acne.

In some embodiments, the duration of treatment is about one day, about one week, about two weeks, about three weeks, about four weeks, about five weeks, about six weeks, about seven weeks, about eight weeks, about nine weeks, about 10 weeks, about 11 weeks, about 12 weeks, about 13 weeks, about 14 weeks, about 15 weeks, about 16 weeks, about 17 weeks, about 18 weeks, about 19 weeks, about 20 weeks, about 24 weeks, about 30 weeks, about 36 weeks, about 40 weeks, about 48 weeks, about 50 weeks, about one year, about two years, about three years, about four years, about five years, or as needed based on the appearance of symptoms of acne. In preferred embodiments, duration of treatment is about 12 weeks to about 24 weeks, about 12 to about 36 weeks, about 12 to about 48 weeks, or about 24 to about 36 weeks.

Also provided in another aspect is a regime or regimen for reducing the number of acne lesions, comprising administering to an individual afflicted therewith an effective amount of any one of the compositions described herein.

In some embodiments, the acne lesions being of inflammatory and/or non-inflammatory type. In some embodiments, the acne lesions are inflammatory type. In some embodiments, the acne lesions are non-inflammatory type.

Provided in another aspect, is a regimen for treatment of acne vulgaris, comprising,
i) cleaning skin;
ii) applying a topically applicable composition according any one of the compositions described herein to the skin; and
iii) applying a moisturizing treatment to the skin.

Provided in another aspect, is a regimen for treatment of acne vulgaris, comprising,
i) cleaning skin; and
ii) applying a topically applicable composition according any one of the compositions described herein to the skin.

In some embodiments, regimen for treatment of acne vulgaris comprising administering said topically applicable composition for at least twenty-four (24) months. In some embodiments, regimen for treatment of acne vulgaris comprising administering said topically applicable composition for at least twelve (12) months. In some embodiments, regimen for treatment of acne vulgaris comprising administering said topically applicable composition for at least nine (9) months. In some embodiments, regimen for treatment of acne vulgaris comprising administering said topically applicable composition for at least six (6) months. In some embodiments, regimen for treatment of acne vulgaris comprising administering said topically applicable composition for at least three (3) months. In some embodiments, regimen for treatment of acne vulgaris comprising administering said topically applicable composition for at least two (2) months. In some embodiments, regimen for treatment of acne vulgaris comprising administering said topically applicable composition for at least one (1) month.

In some embodiments, the regimen for the treatment of acne vulgaris comprises administering said topically applicable composition once a day. In some embodiments, the regimen for the treatment of acne vulgaris comprises administering said topically applicable composition twice a day. In some embodiments, the regimen for the treatment of acne vulgaris comprises administering said topically applicable composition three times a day. In some embodiments, the regimen for the treatment of acne vulgaris comprises administering said topically applicable composition four times a day. In some embodiments, the regimen for the treatment of acne vulgaris comprises administering said topically applicable composition five times a day.

In some embodiments, the regimen for the treatment of acne vulgaris comprises administering said topically applicable composition every two (2) days. In some embodiments, the regimen for the treatment of acne vulgaris comprises administering said topically applicable composition every three (3) days. In some embodiments, the regimen for the treatment of acne vulgaris comprises administering said topically applicable composition every four (4) days. In some embodiments, the regimen for the treatment of acne vulgaris comprises administering said topically applicable composition every five (5) days. In some embodiments, the regimen for the treatment of acne vulgaris comprises administering said topically applicable composition every six (6) days.

In some embodiments, the regimen for the treatment of acne vulgaris comprises administering said topically applicable composition in the morning after washing said affected skin area. In some embodiments, the regimen for the treatment of acne vulgaris comprises administering said topically applicable composition in the evening after washing said affected skin area.

In some embodiments, the affected skin area containing from 20 to 100 non-inflammatory lesions, 20 to 50 inflammatory lesions, and no active nodules or cysts. In some embodiments, the affected skin area contains from 20 to 100 non-inflammatory lesions. In some embodiments, the affected skin area contains from 20 to 50 inflammatory lesions. In some embodiments, the affected skin area contains no active nodules or cysts.

Also provided in another aspect is a regime or regimen for controlling breakouts, comprising administering to an individual afflicted therewith an effective amount of any one of the compositions described herein. In some embodiments, controlling breakouts includes targeting at least one cause of blemishes and blackheads, including but not limited to purifying and cleansing the skin, un-clogging pores, reducing oil, and hydrating skin. In some embodiments, controlling breakouts includes targeting all four causes of blemishes.

### Kits

Provided in one aspect is a kit comprising

a) any one of the topically applicable compositions described herein; and

b) a topical cleanser and/or a topical moisturizer.

In some embodiments, the facial cleaner and/or the facial moisturizer comprise salicylic acid. In some embodiments, the salicylic acid is present in an amount from about 0.1% to about 10%. In some embodiments, the salicylic acid is present in an amount from about 0.1% to about 5%. In some embodiments, the salicylic acid is present in an amount from about 0.1 to about 2.5%. In some embodiments, the salicylic acid is present in an amount of about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 6%, about 7%, about 8%, about 9%, or about 10%. In some embodiments, the salicylic acid is present in an amount of about 0.5%. In some embodiments, the salicylic acid is present in an amount of about 2%.

In some embodiments, the facial cleaner and/or the facial moisturizer comprise benzoyl peroxide. In some embodiments, the benzoyl peroxide is present in an amount from about 0.1% to about 10%. In some embodiments, the benzoyl peroxide is present in an amount from about 0.1% to about 5%. In some embodiments, the benzoyl peroxide is present in an amount from about 0.1 to about 2.5%. In some embodiments, the benzoyl peroxide is present in an amount of about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 6%, about 7%, about 8%, about 9%, or about 10%. In some embodiments, the benzoyl peroxide is present in an amount of about 0.5%. In some embodiments, the benzoyl peroxide is present in an amount of about 2%.

In some embodiments, the facial cleaner and/or the facial moisturizer comprise adapalene. In some embodiments, the adapalene is present in an amount from about 0.1% to about 10%. In some embodiments, the adapalene is present in an amount from about 0.1% to about 5%. In some embodiments, the adapalene is present in an amount from about 0.1% to about 3%. In some embodiments, the adapalene is present in an amount from about 0.1 to about 2.5%. In some embodiments, the adapalene is present in an amount of about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 6%, about 7%, about 8%, about 9%, or about 10%. In some embodiments, the adapalene is present in an amount of about 1%.

### Preparation of Isopropylcarbonate Benzoyl Peroxide Gel Compositions

The Isopropylcarbonate Benzoyl Peroxide compositions described herein may be described in a variety of methods as described in the Examples. This disclosure recognizes that for homogenous dispersion of Isopropylcarbonate Benzoyl Peroxide in the active phase that the addition of the pro-penetrating agent, such as propylene glycol, and a surfactant, such as docusate sodium are important. The addition of these agents improved the “wet-ability” of the aqueous part of the active phase and also reduced undesired foaming.

In Process 1, Phase A is prepared by weighing into a beaker the following components: the first portion of vehicle (such as water), Isopropylcarbonate Benzoyl Peroxide, the opacifier (such as titanium dioxide), liquid wetting surfactant (such as Poloxamer 124), and pro-penetrating agent (such as propylene glycol). These components of Phase A are then mixed together by stirring with a Silverson machine. Phase B is prepared by weighing into a separate beaker the following components: the second portion of vehicle (such as water), sequestering agent (such as disodium EDTA), surfactant (such as docusate sodium), and humectant (such as glycerol). The components of Phase B are dissolved with stirring. With stirring, Phase A is added to Phase B. Then rinsing water (of Phase A vessel and agitator) is added with stirring to the mixture containing Phase A and Phase B. Then the preserving agent (such as phenoxyethanol) and the gelling agent are added to the mixture with stirring. Process 1 provided a very fine dispersion of the opacifier and Isopropylcarbonate Benzoyl Peroxide; however, the dispersion phase is difficult to implement as the mixture thickened and took on the appearance of shaving cream.

Process 2 was developed to prepare a more easily dispersible active phase by reducing the quantity of water in the active phase, adding the surfactant (docusate sodium) in the active phase to increase wetting ability, and introducing the opacifier during the principal phase at the beginning of the process. For Process 2, Phase A is prepared by weighing into a beaker the following components: the first portion of vehicle (such as water), surfactant (such as docusate sodium), liquid wetting surfactant (such as poloxamer 124), and pro-penetrating agent (such as propylene glycol). The surfactant is then dissolved with stirring and then the Isopropylcarbonate Benzoyl Peroxide is added to Phase A. The resulting mixture of Phase A is then further mixed with a Silverson stirring machine. Phase B is prepared by weighing into a separate beaker the following components: the second portion of vehicle (such as water), sequestering agent (such as disodium EDTA), opacifier (such as titanium dioxide), and humectant (such as glycerol). The components of Phase B are dispersed with stirring. With stirring, Phase A is added to Phase B. Then rinsing water (of Phase A vessel and agitator) is added with stirring to the mixture containing Phase A and Phase B. Then the preserving agent (such as phenoxyethanol) and the gelling agent are added to the mixture with stirring. Process 2 provided a fine dispersion of the opacifier into the water of the principle phase. The dispersion phase is easy to implement and with stirring from a Silverson machine, the mixture was fluid and homogeneous. Care was required to limit/control the amount of foam generated from the presence of the surfactant (docusate sodium) in the phase.

A process compatible for large scale production in a Magicplan reactor was developed. Several problems were encountered: Silverson dispersion is not effective because the phase lacks wetting agents; and the final product is bubbly due to air being incorporated either through the dispersion phase or in the principal tank. The process for the Magicplan reactor was modified by introducing the opacifier into the principal tank at the beginning of the process; adding a fraction of the gelling agent at the beginning of the process in order to increase shearing and avoid foaming; adding glycerol at the dispersion phase in order to improve wettability of the Isopropylcarbonate Benzoyl Peroxide; and adding the surfactant (such as docusate sodium) dissolved in water at the end of the process to avoid foaming. Phase A is prepared with following components: the first portion of vehicle (such as water), Isopropylcarbonate Benzoyl Peroxide, humectant (glycerol), liquid wetting surfactant (such as Poloxamer 124), and pro-penetrating agent (such as propylene glycol). Phase B comprises the second portion of the vehicle (such as water), sequestering agent (such as disodium EDTA), the opacifer (such as titanium oxide) and a fraction of the gelling agent. Phase C which contains a third portion of water and surfactant (such as docusate sodium) is added at the end of the process prior to the addition of the preserving agent and a second portion of gelling agent.

In a process suitable for large scale manufacturing, the Isopropylcarbonate Benzoyl Peroxide composition is made by preparing the main phase, which comprising charging a first portion of vehicle (such as water) and the sequestering agent (such as disodium EDTA) into the main tank following by homogenization at a low speed (speed: 40 rpm; emulsifier: 4000 rpm; and time: 15 min). The active phase containing the Isopropylcarbonate Benzoyl Peroxide dispersion (or disaggregation) is prepared by weighing the Isopropylcarbonate Benzoyl Peroxide, a second portion of water, pro-penetrating agent (propylene glycol), humectant (such as glycerol), liquid wetting surfactant (such as poloxamer 124) in a secondary vessel. The secondary vessel is then placed in an ice bath and is then stirred/homogenized slowly to minimize foaming (stirring/homogenization with a Silverson machine; speed: 9000 rpm; and time: 10 mins). The opacifier (such as titanium dioxide) is then added to the main tank and homogenized (speed: 40 rpm; emulsifier: 4000 rpm; and time: 10 min). A first portion of the gelling agent (such as 1% SIMULGEL™ 600 PHA) is then added to the main tank and then dispersed (speed: 70 rpm; emulsifier: 9000 rpm; and time: 10 min). Then the active phase containing the Isopropylcarbonate Benzoyl Peroxide is transferred into the main phase and homogenized (speed: 70 rpm; emulsifier: 9000 rpm; and time: 5 min). The vessels and agitators used for the preparing the Isopropylcarbonate Benzoyl Peroxide are rinsed with rinsed water, which is then added into the main phase. The preserving agent (such as phenoxyethanol) is then added into the main phase (speed: 70 rpm; emulsifier: 9000 rpm; and time: 5 min). In a secondary vessel with magnetic stirring, the surfactant (such as docusate sodium) is dissolved in water (temperature: 40° C.; speed: 400 rpm; and time: 35 min). The dissolved surfactant is then transferred into the main phase with gentle mixing in order to avoid foam formation (speed: 70 rpm; emulsifier: 9000 rpm; and time: 5 min). Finally, the second portion of the gelling agent is then added to the main phase and the stirring speed is increased slowly to prevent foaming (speed: 120 rpm; emulsifier: 2000 rpm; and time: 30 min).

Provided in another aspect is a method for producing an Isopropylcarbonate Benzoyl Peroxide aqueous gel, which method comprises the steps of:
i) solubilizing a sequestering agent in aqueous solution to produce the main phase;
ii) preparing a disaggregation medium comprising Isopropylcarbonate Benzoyl Peroxide by dispersing Isopropylcarbonate Benzoyl Peroxide, propylene glycol, glycerol and liquid wetting surfactant in water to produce medium A; and
iii) adding titanium dioxide to the main phase;
iv) adding a first portion of gelling agent to the main phase;
v) adding medium A to the main phase;
vi) adding of phenoxyethanol to the main phase;
vii) solubilizing docusate sodium in water to provide solubilized docusate sodium followed by addition of the solubilized docusate sodium to the main phase by gentle agitation; and
viii) adding a second portion of gelling agent to the main phase, whereby a gel is formed.

The crystal size of the Isopropylcarbonate Benzoyl Peroxide may be reduced by using a colloid mill. Reducing the crystal size of the Isopropylcarbonate Benzoyl Peroxide provided a homogeneous dispersion with less than about 100 μm. In some embodiments, the Isopropylcarbonate Benzoyl Peroxide from step ii) is subjected to a colloid mill. In some embodiments, subjecting the Isopropylcarbonate Benzoyl Peroxide to a colloid mill provides a homogeneous dispersion that has Isopropylcarbonate Benzoyl Peroxide with the particle size of less than about 100 μm, less than about 90 μm, less than about 80 μm, less than about 70 μm, less than about 60 μm, less than about 55 μm, or less than about 50 μm. In some embodiments, subjecting the Isopropylcarbonate Benzoyl Peroxide to a colloid mill provides a homogeneous dispersion that has Isopropylcarbonate Benzoyl Peroxide with the particle size of about 1 μm to about 100 μm, about 1 μm to about 90 μm, about 1 μm to about 80 μm, about 1 μm to than about 70 μm, about 1 μm to about 60 μm, about 1 μm to about 55 μm, or about 1 μm to about 50 μm.

### EXAMPLES

As referenced in the following examples, CD08467 is Isopropylcarbonate Benzoyl Peroxide. AT as referenced in the following examples refers to ambient temperature. NR as referenced below refers to not reported and RAS as referenced below refers to nothing to report, expected result. Time points such as T0 refers to initial time, T1.5M refers to 1.5 months, T3M refers to 3 months, and T6M refers to 6 months.

Composition A as referenced in the following examples refers to the formulation containing an active agent, such as Isopropylcarbonate Benzoyl Peroxide, and the following components:

TABLE 1

| | | COMPOSITION |
|---|---|---|
| COMPONENT | FUNCTION | A AMOUNT (%) |
| PURIFIED WATER | VEHICLE | 85.025 |
| TITRIPLEX ® III | SEQUESTERING | 0.100 |
| (DISODIUM EDETATE) | AGENT | |
| PROPANEDIOL-1, | PRO-PENETRATING | 4.000 |
| 2 (PROPYLENE GLYCOL) | AGENT | |
| SODIUM DOCUSATE | SURFACTANT | 0.050 |
| GLYCERINE 4810 | HUMECTANT | 4.000 |
| VEGETABLE | | |
| (GLYCEROL) | | |
| KOLLISOLV ® P 124 | LIQUID WETTING | 0.200 |
| (POLOXAMER 124) | SURFACTANT | |
| CD08467 | ACTIVE | 2.625 |
| | INGREDIENT | |
| SIMULGEL ™ 600 PHA | GELLING AGENT | 4.000 |
| (ACRYLAMIDE, AMPS | | |
| COPOLYMERE | | |
| DISPERSION 40%/ | | |
| ISOHEXADECANE) | | |

### Example 1: Solubility in Sebum

In the case of active principles in dispersed form, these can dissolve after application, either in the sebum or in the non-volatile part of the formulation. The maximum solubility of BPO (benzoyl peroxide) and Isopropylcarbonate Benzoyl Peroxide was measured in the following:
The non-volatile part of the Composition A vehicle gel (22 H agitation)
The liquid fraction of the reconstituted sebum (22 H agitation)
The liquid fraction of the reconstituted sebum (extemporaneous)
The sebum reconstituted at 32° C. (22 H agitation)

The results are summarized in the following table and demonstrate that Isopropylcarbonate Benzoyl Peroxide exhibits a solubility profile that is similar to that of benzoyl peroxide:

TABLE 2

| | | | | Liquid |
|---|---|---|---|---|
| | TA | TA | TA | sebum |
| | non- | reconstituted | reconstituted | reconstituted |
| | volatile | liquid sebum | liquid sebum | at 32° C. |
| | (22 H) | (22 H) | (extemporaneous) | (22 H) |
| CD08467 | 0.25% | 1.6% | 0.25% | 1.6% |
| BPO | 0.35% | 1.4% | 1.1% | 1.8% |

### Example 2: Initial Isopropylcarbonate Benzoyl Peroxide Formulations

The following formulations containing 2.5%, 5%, and 10% Isopropylcarbonate Benzoyl Peroxide were prepared.

TABLE 3

| | Composition % w/w-Formula No. | | |
|---|---|---|---|
| Description | BP0158.0001 | BP0158.0002 | BP0158.0003 |
| CD08467 | 2.5 | 5 | 10 |
| PURIFIED WATER | 85.15 | 82.65 | 77.65 |
| GLYCERINE 4810 | 4 | 4 | 4 |
| PLANT | | | |
| KOLLISOLV ® P 124 | 0.2 | 0.2 | 0.2 |
| PROPANEDIOL-1, 2 | 4 | 4 | 4 |
| SIMULGEL ™ 600 PHA | 4 | 4 | 4 |
| SODIUM DOCUSTE | 0.05 | 0.05 | 0.05 |
| SALT | | | |
| TITRIPLEX ® III | 0.1 | 0.1 | 0.1 |

Physical stability

| | | | | |
|---|---|---|---|---|
| Macroscopic | T0 | White gel | White gel | White gel |
| appearance | T1.5M | RAS/NR/viscous | NR | NR |
| TA/30° C./40° C. | | off-white gel | | |
| | T3M | RAS/RAS/ | RAS/RAS/ | RAS/RAS/ |
| | | viscous, slightly | viscous, slightly | viscous, slightly |
| | | brown gel | brown gel | brown gel |

Chemical stability

| | | | | |
|---|---|---|---|---|
| % in relation to | T0 | 100 (LC 101.8) | 100 (LC 103.7) | 100 (LC 96.6) |
| T0 | T1.5M | 100/NR/95 | NR | NR |
| TA/30° C./40° C. | T3M | NR/NR/85 | NR/NR/95 | NR/NR/94 |

Microbiological stability

| pH of the formula to T0 | 4.62 | NR | |
|---|---|---|---|
| Ph Eur. Criteria A | No | | |
| Ph Eur. Criteria B | No | | |
| USP | Yes | | |
| Burkholderia cepacia | No | | |
| | Balance sheet | | |
| | Formula physically | Formula | Formula physically |
| | stable 3 months | physically | stable 3 months |
| | at TA | stable 3 months | at TA |
| | | at TA | |
| | Coloration, off | Coloration, off | Coloration, off |
| | white to brown | white to brown | white to brown |
| | at 40° C. which | at 40° C. which | at 40° C. which |
| | increases over | increases over | increases over |
| | time. | time. | time. |
| | Chemical | Chemical | Chemical |
| | deterioration at | deterioration at | deterioration at |
| | 40° C. | 40° C. | 40° C. |
| | Formula that does | | |
| | not meet the | | |
| | Eur. Pharma- | | |
| | copoeia | | |
| | microbiological | | |
| | criteria. | | |

The results from the above table demonstrate that the formulations containing 2.5%, 5%, and 10% Isopropylcarbonate Benzoyl Peroxide exhibited a beige to brown color at 40° C., where this coloration increases over time. The formulation containing 2.5% Isopropylcarbonate Benzoyl Peroxide did not meet the criteria A and B of the European Pharmacopoeia preservative efficacy test.

### Example 3: Isopropylcarbonate Benzoyl Peroxide Formulations with Preserving Agents

The following preserving agents were tested in the formulations containing 2.5% Compound 1.

TABLE 4

| | Composition % w/w-Formula No. | | | |
|---|---|---|---|---|
| Description | BP0158.0004 | BP0158.0005 | BP0158.0006 | BP0158.0007 |
| CD08467 | 2.5 | 2.5 | 2.5 | 2.5 |
| PURIFIED | 85.05 | 84.35 | 84.15 | 84.95 |
| WATER | | | | |
| GLYCERINE | 4 | 4 | 4 | 4 |
| 4810 PLANT | | | | |
| KOLLISOLV ® P | 0.2 | 0.2 | 0.2 | 0.2 |
| 124 | | | | |
| PROPANEDIOL- | 4 | 4 | 4 | 4 |
| 1,2 | | | | |
| SIMULGEL ™ | 4 | 4 | 4 | 4 |
| 600 PHA | | | | |
| SODIUM | 0.05 | 0.05 | 0.05 | 0.05 |
| DOCUSTE SALT | | | | |
| TITRIPLEX ® III | 0.1 | 0.1 | 0.1 | 0.1 |
| POTASSIUM | 0.1 | / | / | / |
| SORBATE | | | | |
| PHENOXETOL ™ | | 0.8 | 0.8 | / |
| NIPAGIN ™ M | | | 0.2 | 0.2 |

Physical stability

| | | | | | |
|---|---|---|---|---|---|
| Macroscopic | T0 | White gel | White gel | White gel | White gel |
| appearance | T1M | RAS/Off- | RAS/Off- | RAS/Off- | RAS/Off- |
| RT/30° C./40° C. | | white+/ | white+/ | white+/ | white+/ |
| | | Brown++ | Brown++ | Brown++ | Brown++ |
| | T2M | Off-white+ | Off-white+ | Off-white+ | RAS/Off- |
| | | Off-white+/ | Off-white+/ | Off-white+/ | white+/ |
| | | Brown++ | Brown++ | Brown++ | Brown++ |
| | T3M | Off-white+/ | Off-white+/ | Off-white+/ | RAS/NR/ |
| | | NR/Brown++ | NR/Brown++ | NR/Brown++ | Brown++ |
| | T12M | / | Off-white+ | / | / |
| pH RT/30° C./40° C. | T0 | 5.5 | 4.75 | 4.55 | 4.50 |
| | T1M | NR/NR/NR | NR/NR/NR | NR/NR/NR | NR/NR/NR |
| | T2M | 5.21/NR/NR | 4.06/NR/3.18 | 3.93/NR/NR | 4.14/NR/3.16 |
| | T3M | 5.06/NR/3.30 | 3.76/NR/2.90 | 3.74/NR/2.81 | 3.80/NR/2.92 |
| Brookfield | T0 | 163000 cP | 170000 cP | 185000 cP | 178000 cP |
| RVDVII viscosity | T1M | NR/155000 cP | NR/163000 cP | NR/180000 cP | NR/178000 cP |
| Needle: Speed 5: | T2M | 162000 cP/ | 172000 cP/ | 187000 cP/ | 189000 cP/ |
| 0.5 rpm RT/40° C. | | 158000 cP | 160000 cP | 189000 cP | 184000 cP |
| | T3M | 158000 cP/ | 171000 cP/ | 181000 cP/ | 174000 cP/ |
| | | 145000 cP | 171000 cP | 198000 cP | 174000 cP |

Chemical stability

| | | | | | |
|---|---|---|---|---|---|
| % in relation to | T0 | 100 (LC 104.7) | 100 (LC 102.5) | 100 (LC 103.3) | 100 (LC 104.7) |
| T0 RT/30° C./40° C. | T1M | 101.6/NR/ | 100.3/NR/ | 100.7/NR/94 | 99.4/NR/96.9 |
| Series 3 | | 94.65 | 95.5 | | |
| | T2M | 99/NR/89.4 | 100.2/NR/ | 100/NR/85.9 | 98.9/NR/90.4 |
| | | | 90.3 | | |
| | T3M | 98.4/96.1/ | 99.4/97.6/81.8 | 100.8/95.9/ | 98.8/95.8/ |
| | | 82.5 | | 69.4 | 81.6 |
| | T12M | / | 98.4 | / | / |

Microbiological stability

| Ph Eur. Criteria A | NO | YES | YES | YES |
|---|---|---|---|---|
| Ph Eur. Criteria B | NO | YES | YES | YES |
| USP | NO | YES | YES | YES |
| Burkhoderia cepacia | NO | YES | YES | YES |
| | Balance sheet | | | |
| | Formula | Formula | Formula | Formula |
| | physically | physically | physically | physically |
| | stable but | stable but | stable but | stable 3 months |
| | coloration off- | coloration off- | coloration off- | RT, but |
| | white at T2M, | white at T2M, | white at T2M, | coloration off- |
| | increasingly | increasingly | increasingly | white 30, |
| | brown at 40° C. | brown at 40° C. | brown at 40° C. | increasingly |
| | Chemically | Chemically | Formula meets | brown at 40° C. |
| | stable 3 | stable 3 months | microbiologica | Chemically |
| | months at RT | at RT 30° C. but | 1 criteria. | stable 3 months |
| | 30° C. but | deteriorates at | Formula | at RT 30° C. but |
| | deteriorates at | 40° C. at T3M. | chemically | deteriorates at |
| | 40° C. at T2M. | Formula meets | stable at RT | 40° C. at T3M. |
| | Formula that | the Eur. | and 30° C. but | Formula meets |
| | does not meet | Pharmacopoeia | deteriorates at | the |
| | the Eur. | microbiological | 40° C. at T2M | microbiological |
| | Pharma- | criteria, | | criteria; formula |
| | copoeia micro- | | | chemically |
| | bialogical | | | unstable at |
| | criteria. | | | 40° C. |

The results from the above table demonstrate that three of the four preservative systems tested passed the criteria A and B of the European Pharmacopoeia, USP, and Burkhoderia cepacia. Potassium sorbate does not provide adequate antimicrobial protection when used alone. The formulation containing 0.8% phenoxyethanol demonstrated the most desirable stability.

### Example 4: Isopropylcarbonate Benzoyl Peroxide Formulations with Keratolytic Agents

For this study, the following gel based composition was tested:

TABLE 5

| Composition/Formula No.: | 0347.1000 |
|---|---|
| PURIFIED WATER | 94.20% |
| CD08467 | 1.00% |
| SIMULGEL ™ 600 PHA | 4.00% |
| PHENOXETOL ™ | 0.80% |

The following keratolytic agents were tested with the gel based composition indicated above:

TABLE 6

| | | | Compatible | | |
|---|---|---|---|---|---|
| Additives | Chemical name: | Content (%) | Yes | No | Notes |
| GLYCOLIC ACID | GLYCOLIC ACID | 6 | | x | Fall in value 1 m |
| | | | | | 40° C. |
| SALICYLIC ACID | SALICYLIC ACID | 1 | | x | Fall in value 1 m |
| | | | | | 40° C. |
| L-LYSINE | L-LYSINE | 1 | | x | Strong coloration |
| | | | | | 1 m 40° C. |
| COSMACOL ® ECI | TRI-C12-13 ALKY | 6 | | x | Fall in value 1 m |
| | CITRATE | | | | 40° C. |
| COSMACOL ® EMI | DI-C12-13 ALKYL | 5 | | x | Fall in value 1 m |
| | MALATE | | | | 40° C. |
| LACTIC ACID | LACTIC ACID | 1 | x | | |
| GLYCOLIC ACID | GLYCOLIC ACID | 1 | x | | |
| MALIC ACID | MALIC ACID | 1 | x | | |
| PHYTIC ACID | PHYTIC ACID | 1 | x | | |
| SALICYLIC ACID | SALICYLIC ACID | 1 | | x | Fall in value + |
| | | | | | strong coloration |
| | | | | | 3 m 40° C. |
| MICROSILVER | SILVER | 0.05 | x | | |
| BG ™ | | | | | |
| SILVER SOLUTION | SILVER | 2 | x | | |
| B | | | | | |

The results from the above table demonstrate that the following keratolytic agents were compatible: lactic acid, glycolic acid, malic acid, phytic acid, microsilver BG, and silver solution B.

### Example 5: Isopropylcarbonate Benzoyl Peroxide Formulations with Oils

To assess the possibility of a cream formulation that restores the cutaneous barrier, the compatibility of Isopropylcarbonate Benzoyl Peroxide with oils was assessed.

First, the maximum solubilities of Isopropylcarbonate Benzoyl Peroxide in various oils was assessed in the following table.

TABLE 7

| | | | % max sol. | % Max. |
|---|---|---|---|---|
| Commercial name | Chemical Name | Polarity | visual | Solution |
| PRIMOL ™ 352 | MINERAL OIL | Non-polar | 0.41% | 0.1867% |
| PARLEAM ® | HYDROGENATED | Non-polar | 0.50% | 0.1862% |
| | POLYISOBUTENE | | | |
| ST- | CYCLOMETHICONE 5 | Average | 0.33% | 0.1253% |
| CYCLOMETHICONE 5 | | polarity | | |
| NF | | | | |
| SILICON FLUID 20CST | POLYDIMETHYLSILOXANE | Average | 0.18% | 0.0735% |
| | | polarity | | |
| SQUALANE PE | SQUALANE | Non-polar | 0.18% | 0.1781% |
| COS SILKFLO ® 366 | POLYDECENE | Non-polar | 0.18% | 0.1498% |
| ISOSTEARYL | ISOSIEARYL | Average | 1.39% | 0.8679% |
| ISOSTEARATE | ISOSTEARATE | polarity | | |
| MEADOWFOAM SEED | MEADOWFOAM SEED | Polar | 1.96% | 1.2925% |
| OIL | OIL | | | |
| WATER | WATER | Polar | NA | Not measurable |

It is demonstrated from the above table that the solubility of Isopropylcarbonate Benzoyl Peroxide is correlated with the polarity of the oil—the solubility of the Isopropylcarbonate Benzoyl Peroxide increases with increasing polarity of the oil. However, increased solubilization of the Isopropylcarbonate Benzoyl Peroxide also results in greater degradation.

Further the chemical and physical stabilities of the formulations containing 2.5% Isopropylcarbonate Benzoyl Peroxide with various oils were assessed after storage at 3 months RT and 40° C. The value of 2.5% was chosen in order to allow for comparisons to an equivalent formulation containing 2.5% benzoyl peroxide instead of 2.5% Isopropylcarbonate Benzoyl Peroxide.

TABLE 8

| | | Compatible | | |
|---|---|---|---|---|
| Additives | Chemical name: | Yes | No | Notes |
| PRIMOL ™ 352 | MINERAL OIL | x | | Idem BPO |
| PARLEAM ® | HYDROGENATED | x | | Idem BPO |
| | POLYISOBUTENE | | | |
| PRIMOL ™ 352 + | MINERAL OIL + BHT | x | | Idem BPO |
| NIPANOX ™ | | | | |
| ST-CYCLOMETHICONE | CYCLOMETHICONE 5 | | x | Intense orange coloration |
| 5 NF | | | | |
| SILICON FLUID 20 CST | POLYDIMETHYLSILOXANE | | x | Intense orange coloration |
| SQUALANE PE | SQUALANE | x | | |
| COS SILKFLO ® 364 | POLYDECENE | x | | |
| MEADOWFOAM SEED | MEADOWFOAM SEED | | x | Fall in value T1m 40° C. |
| OIL | OIL | | | |
| RUDOL ® WHITE | MINERAL OIL | x | | |
| MINERAL OIL | | | | |
| Q7-9120 SILICONE 20CST + | MIXTURE | x | | slight vs coloration for |
| PRIMOL ™ 352 (90/10) | | | | primol alone |

The results show that Isopropylcarbonate Benzoyl Peroxide is not compatible with silicon oils as this results in an orange coloration and indicates greater chemical degradation as compared to a formulation containing benzoyl peroxide. Isopropylcarbonate Benzoyl Peroxide is also not compatible with meadow foam seed oil. Oils compatible with Isopropylcarbonate Benzoyl Peroxide include, but are not limited to, mineral oil, hydrogenated polyisobutene, squalene, and polydecene.

In order to improve stability, the addition of an anti-oxidant in various selected oils were evaluated.

TABLE 9

| | | | | | | |
|---|---|---|---|---|---|---|
| CD08467 | 2.50% | 2.50% | 2.50% | 2.50% | 2.50% | 2.50% |
| PRIMOL ™ 352 | 97.40% | 97.375% | / | / | / | 97.45% |
| SQUALANE PE | / | / | 97.48% | 97.40% | 97.375% | / |
| NIPANOX ™ BHT | 0.10% | / | 0.02% | 0.10% | / | / |
| DL-ALPHA | / | 0.025% | / | / | 0.025% | / |
| TOCOPHEROL | | | | | | |
| ASCORBYL PALMITATE | / | 0.10% | / | / | 0.10% | / |
| COS MICROSILVER BG ™ | / | / | / | / | / | 0.05% |

% LC Analytical Results

| | | | | | | |
|---|---|---|---|---|---|---|
| T0 | 100.7 | 107.2 | 101 | 100.6 | 91.2 | 106.6 |
| T3M 40° C. | 86.5 | 84 | 76.1 | 81.1 | 73.4 | 70.5 |

The results show that the addition of an anti-oxidant in the selected non-polar oils improves the stability of Isopropylcarbonate Benzoyl Peroxide. Preferred anti-oxidants include, but are not limited to, 0.1% BHT and 0.025% DL alpha tocopherol+0.1% ascorbyl palmitate.

### Example 6: Isopropylcarbonate Benzoyl Peroxide Formulations with Opacifiers

The goal of using an opacifier is to mainly “mask” the evolution of the orange-brown color that appears over time either or the orange color present from T0. The compatibility of Isopropylcarbonate Benzoyl Peroxide with certain components can improve the color stability of the formula, as well as certain feel-related agents. The chemical and physical stabilities were evaluated over 3 months at RT and 40° C.

For this study, the following gel based composition was tested:

TABLE 10

| Composition/Formula No.: | 0347.1000 |
|---|---|
| PURIFIED WATER | 94.20% |
| CD08467 | 1.00% |
| SIMULGEL ™ 600 PHA | 4.00% |
| PHENOXETOL ™ | 0.80% |

The following opacifying agents were tested with the gel based composition indicated above:

TABLE 11

| | Chemical | Content | Compatibility | | |
|---|---|---|---|---|---|
| Additives | name: | (%) | Yes | No | Notes |
| PEARLITE ® 02UVS | BISMUTH | 2 | | x | Fall in value 1 m 40° C. |
| | OXYCHLORIDE | | | | |
| TITANIUM DIOXIDE | TiO₂ | 1 | x | | |
| SUNSHINE ® SOFT | TiO₂ + | 2 | x | | |
| WHITE | FLUORPHLOGO | | | | |
| | PITE | | | | |
| COLORONA ® | MICA/IRON | 0.05 | | x | Fall in viscosity 1 m |
| IMPERIAL CITRINE | OXIDE | | | | 40° C. |
| TIMIRON ® SUPER | MICA/TiO₂ | 2 | x | | |
| SHEEN | | | | | |
| ORGASOL ® 2002 EXT | NYLON 12 | 2 | x | | |
| SunPMMA-S | PMMA | 3 | x | | |
| Orange K7001-J | | 0.003 | | x | Discoloration 1 m |
| | | | | | 40° C. |
| RONAFLAIR ® | BORON | 2 | x | | |
| BORONEIGE ® SPF3 | NITRIDE | | | | |

Based on the results illustrated in the above table, the following opacifying agents appeared to compatible: SunSHINE® soft white, TIMIRON® super sheen, ORGASOL® 2002 EXT, SunPMMA-S, and RONAFLAIR® BORONEIGE® SPF3.

Further optimization studies were performed with the following opacifying agents that were chemically similar:
1% TiO2 (titanium dioxide);
2% WATER BN™ 3002 (Boron Nitride/PEG-8 methyl ether dimethicone);
2% TIMIRON® super sheen MP-1001 (Mica 64%, TiO2 45%); and
3% ORGASOL® 2002 EXD Nat (Nylon12).

The formulations containing the selected opacifers were prepared by stirring in the selected opacifier to a 2.5% Isopropylcarbonate Benzoyl Peroxide gel composition (similar to the 1% Isopropylcarbonate Benzoyl Peroxide gel composition used in the earlier studies). The formulations were monitored for 3 years at RT. The formulations containing opacifier were compared to the corresponding formulations without opacifier in order to evaluate the impact of the opacifier on the decrease in coloration over time.

TABLE 12

| | BP0347.0005 | | | | |
|---|---|---|---|---|---|
| | CD08467 | 0347.PLA | 0347.PLA | 0347.PLA | 0347.PLA |
| Formulation No.: | 2.5% | 20150602/6 | 20150602/7 | 20150602/8 | 20150602/9 |
| Formula | GEL | Manu. | Manu. | Manu. | Manu. |
| composition: | T3ans | 02.06.15 | 02.06.15 | 02.06.15 | 02.06.15 |
| PURIFIED WATER | QS | QS | QS | QS | QS |
| CD08467 | 2.50% | 2.50% | 2.50% | 2.50% | 2.50% |
| TRITRIPLEX ® III | 0.10% | 0.10% | 0.10% | 0.10% | 0.10% |
| SIMULGEL ™ 600 | 4.00% | 4.00% | 4.00% | 4.00% | 4.00% |
| PHA | | | | | |
| PHENOXETOL ™ | 0.80% | 0.80% | 0.80% | 0.80% | 0.80% |
| SODIUM | 0.05% | 0.05% | 0.05% | 0.05% | 0.05% |
| DOCUSATE | | | | | |
| GLYCERIN 4810 | 4.00% | 4.00% | 4.00% | 4.00% | 4.00% |
| KOLLISOLV ® P | 0.20% | 0.20% | 0.20% | 0.20% | 0.20% |
| 124 | | | | | |
| PROPANEDIOL-1,2 | 4.00% | 4.00% | 4.00% | 4.00% | 4.00% |
| TiO₂ (TITANIUM | / | 1.00% | / | / | / |
| DIOXIDE) | | | | | |
| BN ™ 3002 WATER | / | / | 2.00% | / | / |
| (BORON | | | | | |
| NITRIDE/PEG-8 | | | | | |
| METHYL ETHER | | | | | |
| DIMETHICONE) | | | | | |
| TIMIRON ® SUPER | / | / | / | 2.00% | / |
| SHEEN MP-1001 | | | | | |
| (MICA 64%, TIO₂ | | | | | |
| 45%) | | | | | |
| ORGASOL ® 2002 | / | / | / | / | 3.00% |
| EXD NAT | | | | | |
| (NYLON12) | | | | | |
| Notes | Color counter | T0: Gel | T0: Gel | T0: Pearly* | T0: Off-white |
| | type: | slightly off- | slightly off- | off-white gel | to light yellow* |
| | Gel orange | white* mat | white* | T3J: RAS | gel |
| | (deterioration | T3J: RAS | T3J: RAS | pearly* off- | T3J: RAS off- |
| | of active | Gel slightly | Gel | white gel | white to yellow |
| | principle) | off-white* | slightly off- | | gel |
| | → No addition | mat | white* | | |
| | of opacifers | | | | |
| Conclusion | / | Suitable, | Partial | Partial | Coverage: Not |
| | | partial | coverage: Not | coverage: Not | OK |
| | | covering: | OK | OK | |
| | | Opacifying | | | |
| | | agent | | | |
| | | selected OK | | | |

From the results shown above, the only opacifier where the formulation retained desirable coloration is TiO2. It also provided the formulation with adequate “stickiness” when applied.

The concentration of TiO2 was explored in order to reduce to “stickiness” of the formulation. As shown in the below table, the following amounts of TiO2 were evaluated in a 2.5% Isopropylcarbonate Benzoyl Peroxide gel composition: 0.2%, 0.4%, 0.5%, 0.8%, and 1%.

TABLE 13

| | | | | | | |
|---|---|---|---|---|---|---|
| Formulation No.: | BP0347.0006 | 0347.PLA | 0347.PLA | 0347.PLA | 0347.PLA | 0347.PLA |
| Formula | CD08467 5% | 20150616/1 | 20150616/2 | 20150616/3 | 20150616/4 | 20150616/5 |
| composition: | GEL | Manu.: | Manu.: | Manu.: | Manu.: | Manu.: |
| | T3ans | 6/16/2015 | 6/16/2015 | 6/16/2015 | 6/16/2015 | 6/16/2015 |
| | Manu.: | | | | | |
| | 7/24/2012 | | | | | |
| PURIFIED WATER | QS | QS | QS | QS | QS | QS |
| CD08467 | 5.00% | 5.00% | 5.00% | 5.00% | 5.00% | 5.00% |
| TRITRIPLEX ® III | 0.10% | 0.10% | 0.10% | 0.10% | 0.10% | 0.10% |
| SIMULGEL ™ 600 PHA | 4.00% | 4.00% | 4.00% | 4.00% | 4.00% | 4.00% |
| PHENOXETOL ™ | 0.80% | 0.80% | 0.80% | 0.80% | 0.80% | 0.80% |
| SODIUM DOCUSATE | 0.05% | 0.05% | 0.05% | 0.05% | 0.05% | 0.05% |
| GLYCERINE 4810 | 4.00% | 4.00% | 4.00% | 4.00% | 4.00% | 4.00% |
| KOLLISOLV ® P124 | 0.20% | 0.20% | 0.20% | 0.20% | 0.20% | 0.20% |
| PROPANEDIOL-1,2 | 4.00% | 4.00% | 4.00% | 4.00% | 4.00% | 4.00% |
| TiO₂ (TITANIUM DIOXIDE) | / | 0.20% | 0.40% | 0.50% | 0.80% | 1.00% |
| Notes | Color | T0: Light | T0: Gel | T0: Gel | T0: Gel | T0: Gel |
| | counter type: | yellow | slightly | slightly | slightly | slightly |
| | Gel | mat gel | off-white* | off-white* | off-white* | off-white* |
| | orange | | mat | mat | mat | mat |
| | (deterioration | | | | | |
| | of the active | | | | | |
| | principle) | | | | | |
| | → No | | | | | |
| | addition | | | | | |
| | of opacifiers | | | | | |
| Conclusion | / | Opacification: | Suitable, | Suitable, | Suitable, | Suitable, |
| regarding | | not OK | partial | partial | partial | partial |
| opacification | | | covering: OK | covering: OK | covering: OK | covering: OK |

| | |
|---|---|
| Optimal % TiO₂ | Between 1% and 0.4% TiO₂, not significant differences observed in terms of opacification: Slightly off-white gel. |
| | A color difference was observed beginning at 0.2%: light yellow. gel |
| | → The optimized proportion of TiO₂ is 0.4% |

No significant differences was observed between 1% and 0.4% TiO2 in terms of opacification. A color difference was observed beginning at 0.2%. Based on these studies, 0.4% TiO2 was selected as the opacifier for the Isopropylcarbonate Benzoyl Peroxide formulations.

An exemplary Isopropylcarbonate Benzoyl Peroxide formulation containing an opacifier is shown below:

TABLE 14

| | | | Theoretical Quantity |
|---|---|---|---|
| Article description | Consensus name | Function | (g) |
| PURIFIED WATER | PURIFIED WATER | VEHICLE | 76.450000 |
| TITRIPLEX ® III | DISODIUM EDETATE | SEQUESTERING | 0.100000 |
| | | AGENT | |
| VEGETABLE GLYCERINE | GLYCEROL | HUMECTANT | 2.000000 |
| 4810 | | | |
| SODIUM DOCUSATE SALT | SODIUM DOCUSATE | SURFACTANT | 0.050000 |
| KOLUSOLV ® P 124 | POLOXAMER 124 | LIQUID WETTING | 0.200000 |
| | | SURFACTANT | |
| PROPANEDIOL 1,2 | PROPYLENE | PRO-PENETRATING | 4.000000 |
| | GLYCOL | AGENT | |
| GLYCERINE 4810 | GLYCEROL | HUMECTANT | 2.000000 |
| VEGETABLE | | | |
| TITANIUM DIOXIDE | | OPACIFIER | 0.400000 |
| PURIFIED WATER | PURIFIED WATER | VEHICLE | 10.000000 |
| PHENOXETOL ™ | PHENOXYETHANOL | PRESERVING AGENT | 0.800000 |
| SIMULGEL ™ 600 PHA | ACRYLAMIDE, AMPS | GELLING AGENT | 4.000000 |
| | COPOLYMERE | | |
| | DISPERSION 40%/ | | |
| | ISOHEXADECANE | | |

Another alternative to opacify an Isopropylcarbonate Benzoyl Peroxide formulation is to opacify the gel composition with a fatty phase in order to create an emulsion and then whiten the formulation. An exemplary formulation is shown below:

TABLE 15

| | | | Theoretical |
|---|---|---|---|
| Article description | Consensus name | Function | Quantity |
| PURIFIED WATER | PURIFIED WATER | VEHICLE | 81.000000 |
| KOLUSOLV ® P 124 | POLOXAMER 124 | LIQUID WETTING | 0.200000 |
| | | SURFACTANT | |
| PROPANEDIOL 1,2 | PROPYLENE GLYCOL | PRO-PENETRATING | 4.000000 |
| | | AGENT | |
| PHENOXETOL ™ | PHENOXYETHANOL | PRESERVING AGENT | 0.800000 |
| SIMILGEL ™ 600 PHA | ACRYLAMIDE, AMPS | GELLING AGENT | 4.000000 |
| | COPOLYMERE | | |
| | DISPERSION 40%/ | | |
| | ISOHEXADECANE | | |
| COS PARLEAM ® 4 | | EMOLIENT | 6.000000 |
| SQUALANE-PE | | EMOLIENT | 4.000000 |

The above formulation has the advantage of being “very white” with a dry feel and pleasant touch, which are compatible for use in acne.

### Example 7: Isopropylcarbonate Benzoyl Peroxide Formulations with Skin Conditioning Agents

The following skin conditioning agents were tested for their ability to provide a formulation with the following features: soft texture, rapid penetration, a non-sticky residue, and mattified skin after application.

1. 2% Sunsil 150-H (Silica beads)

2. 2% Sun PMMA-S (methyl methacrylate cross polymer)

3. 2% Sun PMMA-X (methyl methacrylate cross polymer)

4. 2 to 3% ORGASOL® 2002 EXD Nat (Nylon12)

5. 0.7 to 2% TIMIRON® super sheen MP-1001 (Mica 64%, TiO2 45%)

6. 0.7 to 2% JH-Gold (mica, polymethyl methacrylate, titanium dioxide)

TABLE 16

| | | | | | | |
|---|---|---|---|---|---|---|
| Formulation No.: | 0347.PLA | 0347.PLA | 0347.PLA | 0347.PLA | 0347.PLA | 0347.PLA |
| Formula composition: | 20150615/1 | 20150615/2 | 20150615/3 | 20150615/4 | 20150615/5 | 20150624/6 |
| | Manu.: | Manu.: | Manu.: | Manu.: | Manu.: | Manu.: |
| | 6/15/2015 | 6/15/2015 | 6/15/2015 | 6/15/2015 | 6/15/2015 | 6/24/2015 |
| PURIFIED WATER | QS | QS | QS | QS | QS | QS |
| SIMULGEL ™ 600 PHA | 4.00% | 4.00% | 4.00% | 4.00% | 4.00% | 4.00% |
| GLYCERINE 4810 | 4.00% | 4.00% | 4.00% | 4.00% | 4.00% | 4.00% |
| KOLLISOLV ® P124 | 0.20% | 0.20% | 0.20% | 0.20% | 0.20% | 0.20% |
| PROPANEDIOL-1,2 | 4.00% | 4.00% | 4.00% | 4.00% | 4.00% | 4.00% |
| TiO₂ (TITANIUM DIOXIDE) | 0.40% | 0.40% | 0.40% | 0.40% | 0.40% | 0.40% |
| Sunsil 150-H | / | 2.00% | / | / | / | / |
| Sun PMMA-S | / | / | 2.00% | / | / | / |
| Sun PMMA-X (METHYL | / | / | / | 2.00% | / | / |
| METHACRYLATE | | | | | | |
| CROSS POLYMER) | | | | | | |
| ORGASOL ® 2002 | / | / | / | / | 2.00% | 3.00% |
| EXD NAT (NYLON12) | | | | | | |
| Notes | Visual | Visual | Visual | Visual | Visual | Visual |
| | appearance: | appearance: | appearance: | appearance: | appearance: | appearance: |
| | Shiny white | Shiny white | Shiny white | Shiny white | Shiny white | Shiny white |
| | gel mat | gel mat | gel mat | gel mat | gel mat | gel mat |
| | Feel upon | Feel upon | Feel upon | Feel upon | Feel upon | Feel upon |
| | application: | application: | application: | application: | application: | application: |
| | Soft, | thick. | Rough, | Thick, sticky | Thick, sticky | Thick, sticky |
| | aqueous | Residual | difficult | movement, | movement. | movement. |
| | Residual feel: | feel: sticky. | penetration. | Residual | Final feel: | Final feel: |
| | sticky. | | Residual | feel: Soft, | Soft, | Soft, |
| | Appearance | | feel: rough. | dry (after a | | |
| | of the skin after | | | waiting period). | | |
| | application: | | | | | |
| Texture conclusion | Texture | Texture: | Texture: | Interesting | Interesting | Interesting |
| | acceptable: OK | Not OK | Not OK | texture: OK | texture: OK | texture: OK |

| | |
|---|---|
| Selected texture agents | According to the results of the sensorial assessment, the formulas that are interesting in terms of texture are: |
| | TiO₂ alone |
| | Sun PMMA-X (2%) |
| | ORGASOL ®: At 3% the texture is slightly more pleasant than at 2% (more marked stickiness) |

From the above table, the selected skin conditioning agents were 0.4% TiO2, 0.2% Sun PMMA-X 2%, and 3% ORGASOL® 2002 EXD NAT. Sun PMMA-X was selected for further studies as its texture-improving properties (final soft feel) and for its mattifying properties.

A second study was performed to screen for powder skin conditioning agents with desirable mattifying properties and improvement of feel. In this study, glycerin was not used in the tested formulation in order to optimize texture.

TABLE 17

| | BP0347.1150P | 0347. PLA | 0347. PLA | 0347. PLA | 0347. PLA |
|---|---|---|---|---|---|
| Formulation No.: | 15.01433 | 20150710/1 | 20150710/2 | 20150710/3 | 20150710/4 |
| Formula | Manu.: | Manu.: | Manu.: | Manu.: | Manu.: |
| composition: | Jul. 10, 2015 | Jul. 10, 2015 | Jul. 10, 2015 | Jul. 10, 2015 | Jul. 10, 2015 |
| PURIFIED WATER | QS | QS | QS | QS | QS |
| SODIUM DOCUSATE | 0.05% | 0.05% | 0.05% | 0.05% | 0.05% |
| SALT | | | | | |
| KOLLISOLV ® P 124 | 0.20% | 0.02% | 0.20% | 0.20% | 0.20% |
| PROPANEDIOL-1,2 | 4.00% | 4.00% | 4.00% | 4.00% | 4.00% |
| SIMULGEL ™ 600 | 4.00% | 4.00% | 4.00% | 4.00% | 4.00% |
| PHA | | | | | |
| PHENOXETOL ™ | 0.80% | 0.80% | 0.80% | 0.80% | 0.80% |
| TiO₂ (TITANIUM | 0.40% | 0.40% | 0.40% | 0.40% | 0.40% |
| DIOXIDE) | | | | | |
| Sun PMMA-X | / | 1.00% | 2.00% | / | / |
| (METHYL | | | | | |
| METHACRYLATE | | | | | |
| CROSS POLYMER) | | | | | |
| Sun PMMA COCO-130 | / | / | / | 1.00% | 2.00% |
| (METHYL | | | | | |
| METHACRYLATE | | | | | |
| CROSS POLYMER) | | | | | |
| Physical appearance/ | Visual | Visual | Visual | Visual | Visual |
| Texture Assessment | appearance: | appearance: | appearance: | appearance: | appearance: |
| | Shiny white | Shiny white | Shiny white | Shiny white gel | Shiny white |
| | gel mat | gel mat | gel mat | mat | mat gel |
| | Feel upon | Feel upon | Feel upon | Feel upon | Feel upon |
| | application: | application: | application: | application: | application: |
| | Soft, aqueous | Thick, sticky | Thick, sticky, | Thick, sticky, | Thick, sticky |
| | Residual feel: | movement | movement. | slow | slow |
| | Stickiness | Residual feel: | Residual feel: | movement | movement |
| | (less than the | Not | Soft, dry (after | Residual feel: | Residual feel: |
| | Placebo with | assessable, no | a waiting | rough. | rough. |
| | glycerin) | effect | period) | Appearance of | Appearance of |
| | Skin | Appearance | Skin | the skin after | the skin after |
| | appearance | of the skin | appearance | application:/ | application:/ |
| | after | after | after | | |
| | application: | application: | application: not | | |
| | shiny | Slightly | shiny, | | |
| | | mattified | mattified | | |
| Texture conclusion | REFERENCE | Test Sun | Test Sun | Test Sun | Test Sun |
| | PLACEBO | PMMA-X | PMMA-X 2%: | PMMA | PMMA |
| | with 0.4%TiO₂ | 1%: Texture | Texture OK | COCO-130 | COCO-130 |
| | | NOT OK | → % minimum: | 1%: Texture | 2%: Texture |
| | | → Significant | 2% | NOT OK | NOT OK |
| | | sensory loss | | | |
| | | from 1% to | | | |
| | | 2% | | | |

| | |
|---|---|
| Selected texture agents | According to the sensory assessment, the texture agent |
| | providing optimum results is: 2% Sun PMMA-X. |

The additional skin conditioning agents were screened:

7. 2% Sunsil 150-H (Silica beads)

8. 2% Sun PMMA-S (methyl methacrylate cross polymer)

9. 2% Sun PMMA-X (methyl methacrylate cross polymer)

10. 2 to 3% ORGASOL® 2002 EXD Nat (Nylon12)

11. 0.7 to 2% TIMIRON® Super Sheen MP-1001 (Mica 64%, TiO2 45%)

12. 0.7 to 2% JH-Gold (mica, polymethyl methacrylate, titanium dioxide)

The selected formulations were:

TABLE 18

| Composition/Formula No. | 0347.1136P | 0347.1137P | 0347.1138P | 0347.1139P |
|---|---|---|---|---|
| PURIFIED WATER | QS | QS | QS | QS |
| TITRIPLEX ® III | 0.10 | 0.10 | 0.10 | 0.10 |
| SIMULGEL ™ 600 PHA | 4.00 | 4.00 | 4.00 | 4.00 |
| PHENOXETOL ™ | 0.80 | 0.80 | 0.80 | 0.80 |
| SODIUM DOCUSATE SALT | 0.05 | 0.05 | 0.05 | 0.05 |
| GLYCERIN 4810 | 4.00 | 4.00 | 4.00 | 4.00 |
| KOLLISOLV ® P 124 | 0.20 | 0.20 | 0.20 | 0.20 |
| PROPANEDIOL-1, 2 | 4.00 | 4.00 | 4.00 | 4.00 |
| TiO₂ | 0.40 | 0.40 | 0.40 | 0.40 |
| ORGASOL ® 2002 EXD NAT | / | 3.00 | / | / |
| Sun PMMA-X | / | / | 2.00 | / |
| JH-Gold | / | / | / | 0.70 |

The physical and chemical stability of the following formulations were evaluated:

TABLE 19

| Composition/Formula | 0347.1108 | | | | |
|---|---|---|---|---|---|
| No. | Reference | 0347.1156 | 0347.1157 | 0347.1158 | 0347.1151 |
| PURIFIED WATER | QS | QS | QS | QS | QS |
| SIMULGEL ™ 600 | 4.00 | 4.00 | 4.00 | 4.00 | 4.00 |
| PHA | | | | | |
| PHENOXETOL ™ | 0.80 | 0.80 | 0.80 | 0.80 | 0.80 |
| KOLLISOLV ® P 124 | 0.20 | 0.20 | 0.20 | 0.20 | 0.20 |
| PROPANEDIOL-1, 2 | 4.00 | 4.00 | 4.00 | 4.00 | 4.00 |
| TiO₂ | / | 0.40 | 0.40 | 0.40 | / |
| COS PARLEAM ® 4 | / | / | / | / | 6 |
| SQUALANE-PE | / | / | / | / | 4 |
| Sun PMMA-X | / | / | / | 2.00 | / |
| JH-Gold | / | / | 0.70 | / | / |
| CD08467 | 1 | 1 | 1 | 1 | 1 |
| Physical Appearance | Shiny white gel | Shiny white | Pearlized gel, | Shiny white | Thick white |
| | | opaque gel | hints of gold | opaque gel | cream |
| T3M | Shiny white gel/ | Shiny white gel/ | Shiny white gel/ | Shiny white gel | Shiny, very |
| TA/30° C./40° C. | very slightly | RAS/slightly | slightly off- | slightly off- | slightly off- |
| | off-white gel/ | beige | white/off- | white/off- | white cream/ |
| | beige | coloration | white | white | 30° C./ |
| | coloration | | coloration | coloration | slightly |
| | | | | | orange/brown |
| | | | | | shiny cream |
| pH T0 | 4.71 | 4.39 | 4.63 | 4.45 | 4.78 |
| T3M TA/30° C./40° C. | 3.90/3.33/2.95 | 3.74/3.37/2.92 | 4.15/3.46/3.01 | 3.93/3.35/2.85 | 3.60/2.85 |
| T6M TA/30° C./40° C. | | | | | 3.59/2.81 |
| Viscosity | RV, Needle 6, | RV, Needle 6, | RV, Needle 6, | RV, Needle 6, | RV, Needle |
| TA/30° C./40° C. | Speed = 5 | Speed = 5 | Speed = 5 | Speed = 5 | 6, Speed =10 |
| | 105,000 cP | 109,000 cP | 120,000 cP | 134,000 cP | 72400 cP |
| | T3M | T3M | T3M | T3M | T3M |
| | 110000/107000/ | 106000/106000/ | 122000/115000/ | 147000/148000 | 69700/32000 |
| | 57000 | 60000 | 51000 | 90000 | T6M |
| | | | | | 66000/18100 |
| Notes | | Stability | Stability | Stability | Stability |
| | | equivalent to | equivalent to | equivalent to | equivalent to |
| | | the reference | the reference | the reference | the reference |

TABLE 20

| | T0 | | | T1M | T2M | T3M |
|---|---|---|---|---|---|---|
| 0347.1108/15.01606 | 100 | TA | % / T0 | 102.9 | 97.5 | 98.4 |
| 1% CD08467 | (101.6) | 30° C. | % / T0 | — | / | 90.9 |
| Simplified gel base (4% SIMULGEL ET 0.8% | | 40° C. | % / T0 | 89.7 | 69.7 | 35.8 |
| PHENOX) + KOLLISOLV ® P24 + PG | | | | | | |
| 0347.1156/15.01639 | 100 | TA | % / T0 | 99.6 | 98.6 | 98.2 |
| 1% CD08467 | (103.5) | 30° C. | % / T0 | — | / | 96.4 |
| Simplified gel base (4% SIMULGEL AND 0.8% | | 40° C. | % / T0 | 90.9 | 70.2 | 39.0 |
| PHENOX) + KOLLISOLV ® p24 + PG + 0.4% TiO₂ | | | | | | |
| 0347.1157/15.01641 | 100 | TA | % / T0 | 100.2 | 100.0 | 99.5 |
| 1% CD08467 | (106.5) | 30° C. | % / T0 | — | / | 92.5 |
| Simplified gel base (4% SIMULGEL AND 0.8% | | 40° C. | % / T0 | 92.6 | 69.4 | 37.2 |
| PHENOX) + KOLLISOLV ® P24 + PG + 0.4% | | | | | | |
| TiO₂ + 0.7% JH-Gold | | | | | | |
| 0347.1158/15.01644 | 100 | TA | % / T0 | 99.7 | 99.1 | 98.2 |
| 1% CD08467 | (101.1) | 30° C. | % / T0 | — | / | 90.9 |
| Simplified gel base (4% SIMULGEL and 0.8% | | 40° C. | % / T0 | 89.7 | 68.5 | 40.8 |
| PHENOX) + KOLLISOLV ® P24 + PG + 0.4% | | | | | | |
| TiO₂ + 2% Sun PMMA-X | | | | | | |
| 0347.1151/15.01457 | 100 | TA | % / T0 | 100.1 | 101.6 | 101.1 |
| 1% CD08467 | (102.6) | | | | | |
| Emulsion (PARLEAM ® 4-6% / SQUALANE PE | | | | | | |
| 4%) without EDTA (4% SIMULGEL and 0.8% | | | | | | |
| PHENOX) + KOLLISOLV ® P24 + PG | | | | | | |

The above formulation have chemical and physical stabilities equivalent to the reference formulation. The reduction in undesirable coloration is greatly improved compared to the reference formulation.

### Example 8: Isopropylcarbonate Benzoyl Peroxide Formulations with Gelling Agents

In these studies, the gelling agents used in the Isopropylcarbonate Benzoyl Peroxide formulations was explored. The first optimization project was to substitute SIMULGEL™ 600 PHA with another gelling agent (or gelling system) which could improve the sensory aspects of the formula. This new gelling agent could also have a positive impact on physical and chemical stability (color, viscosity).

The SEPPIC range of gelling agents were explored. The gelling agents were used to obtain formulations with viscosities equivalent to that of SIMULGEL™ 600 PHA at 4%.

TABLE 21

| Gelling | SEPIPLUS ™ | | SIMULGEL ™ | SEPINOV ™ | SEPIMAX |
|---|---|---|---|---|---|
| Agent | 400 | SEPIPLUS ™ S | INS 100 | EMT 10 | ZEN ™ |
| Nom INCI | Polyacrylate- | Hydroxyethyl | Hydroxyethyl | Hydroxyethyl | Polyacrylate |
| | 13 & | Acrylate/Sodium | Acrylate/ | Acrylate/ | Crosspolymer- |
| | Polyisobutene | Acryloyldimethyl | Sodium | Sodium | 6 |
| | & Polysorbate | Taurate Copolymer | Acryloyldimethyl | Acryloyldimethyl | |
| | 20 | & Polyisobutene & | Taurate | Taurate | |
| | | PEG-7 | Copolymer & | Copolymer | |
| | | Trimethylolpropane | Isohexadecane & | | |
| | | Coconut ether | Polysorbate 60 | | |
| Supplier | Seppic | Seppic | Seppic | Seppic | Seppic |
| Preservative | None | None | None | None | None |
| System | | | | | |
| Properties | Thickening, | Thickening, | Thickening, | Thickening, | Thickening, |
| | stabilizing, | stabilizing, use | stabilizing, use | stabilizing, use | stabilizing, |
| | use pH = 3 − | pH = 3 − 11 | pH = 3 − 11, | pH = 3 − 12, | use pH = 2 − 8, |
| | 11, Effective | | development of | Effective at low | makes it |
| | at low dose. | | all types of | dose, makes it | possible to |
| | | | consistencies: | possible to obtain | obtain |
| | | | Sprayable, ultra- | transparent gels. | transparent |
| | | | fluid to very | | gels. |
| | | | thick | | |

Furthermore, these formulations were then prepared with 1% active principle and compared to a SIMULGEL™ 600 reference.

TABLE 22

| Composition No.: | | | | | | | |
|---|---|---|---|---|---|---|---|
| Formula | 0347.0020 | 0347.1173 | 0347.1174 | 0347.1175 | 0347.1176 | 0347.1177 | 0347.1178 |
| INGREDIENT | % w/w | | | | | | |
| PURIFIED WATER | QSP 100 | QSP 100 | QSP 100 | QSP 100 | QSP 100 | QSP 100 | QSP 100 |
| TITRIPLEX ® III | 0.1 | 0.1 | 0.1 | 0.1 | 0.1 | 0.1 | 0.1 |
| GLYCERIN 4810 | 4 | 4 | 4 | 4 | 4 | 4 | 4 |
| KOLLISOLV ® P124 | 0.2 | 0.2 | 0.2 | 0.2 | 0.2 | 0.2 | 0.2 |
| PROPANEDIOL-1,2 | 4 | 4 | 4 | 4 | 4 | 4 | 4 |
| SODIUM DOCUSAIL | 0.05 | 0.05 | 0.05 | 0.05 | 0.05 | 0.05 | 0.05 |
| PHENOXETOL ™ | 0.8 | 0.8 | 0.8 | 0.8 | 0.8 | 0.8 | 0.8 |
| SIMULGEL ™ 600 PHA | 4 | — | — | — | — | — | — |
| SEPIPLUS ™ 400 | — | 3 | — | — | — | 2.85 | — |
| SEPIPLUS ™ S | — | — | 3.25 | — | — | — | — |
| SEPINOV ™ EMT10 | — | — | — | 2.2 | — | — | — |
| SEPIMAX ZEN ™ | — | — | — | — | 3.5 | — | — |
| SATIAXANE ™ UCX 911 | — | — | — | — | — | 0.5 | — |
| SIMULGEL ™ INS100 | — | — | — | — | — | — | 4.5 |
| CD08467 | 1 | 1 | 1 | 1 | 1 | 1 | 1 |

TABLE 23

| | T0 | | | T1M | T2M | T3M | T6M |
|---|---|---|---|---|---|---|---|
| 0347.0020 | | TA | % / T0 | — | 104.3 | 98.3 | |
| 1% CD08467 | 100.8 | 30° C. | % / T0 | — | | 92.3 | |
| Gel formula (SIMULGEL ™ 600) | (100) | 40° C. | % / T0 | 90.4 | 79.8 | 53.7 | |
| 0347.1173 | | TA | % / T0 | — | Halted due to physical | | |
| 1% CD08467 | 94.6 | 30° C. | % / T0 | — | stability problem (color, | | |
| Gel formula SEPIPLUS ™ 400 | (100) | 40° C. | % / T0 | 91.4 | viscosity) | | |
| 0347.1174 | | TA | % / T0 | — | | | |
| 1% CD08467 | 98.9 | 30° C. | % / T0 | — | | | |
| Gel formula SEPIPLUS ™ S | (100) | 40° C. | % / T0 | 91.2 | | | |
| 0347.1175 | | TA | % / T0 | — | 105.7 | 99.5 | |
| 1% CD08467 | 98.9 | 30° C. | % / T0 | — | | 94.3 | |
| Gel formula SEPINOV ™ EMT10 | (100) | 40° C. | % / T0 | 92.0 | 79.1 | 55.7 | |
| 0347.1176 | | TA | % / T0 | — | Halted due to physical | | |
| 1% CD08467 | 92.7 | 30° C. | % / T0 | — | stability problem (color, | | |
| Gel formula SEPIMAX ZEN ™ | (100) | 40° C. | % / T0 | 91.6 | viscosity) | | |
| 0347.1177 | | TA | % / T0 | — | | | |
| 1% CD08467 | 97.7 | 30° C. | % / T0 | — | | | |
| Gel formula SEPIPLUS ™ 400 | (100) | 40° C. | % / T0 | 92.5 | | | |
| 0347.1178/15.02435 Fable | | TA | % / T0 | — | 103.9 | 99.1 | |
| 12/1/2015 | | | | | | | |
| 1% CD08467 | 97.2 | 30° C. | % / T0 | — | | 95.7 | |
| Gel formula SIMULGEL ™ INS 100 | (100) | 40° C. | % / T0 | 93.9 | 79.5 | 62.2 | |

The SEPINOV™ EMT 10 and SIMULGEL™ INS 100 gelling agents provided formulations that were chemically and physically stable.

### Example 9: Isopropylcarbonate Benzoyl Peroxide Formulations with and without TiO2

Starting with a gel formulation containing water, SIMULGEL™ 600, and phenoxyethanol, components were successively incorporated to obtain a complete formulation.

TABLE 24
Series without TiO2

| | 0347.1000/ | 0347.1108/ | 0347.1179/ | 0347.1181/ | 0347.0020/ |
|---|---|---|---|---|---|
| | 15.02483 | 15.02495 | 15.02503 | 15.02505 | 15.02509 |
| INGREDIENT | % w/w | | | | |
| PURIFIED | QSP100 | QSP100 | QSP100 | QSP100 | QSP100 |
| WATER | | | | | |
| CD08467 | 1 | 1 | 1 | 1 | 1 |
| SIMULGEL ™ | 4 | 4 | 4 | 4 | 4 |
| 600 | | | | | |
| PHENOXETOL ™ | 0.8 | 0.8 | 0.8 | 0.8 | 0.8 |
| KOLLISOLV ® | — | 0.2 | 0.2 | 0.2 | 0.2 |
| P124 | | | | | |
| PROPANEDIOL | — | 4 | 4 | 4 | 4 |
| GLYCERIN 4810 | — | — | 4 | 4 | 4 |
| TITRIPLEX ® III | — | — | — | 0.1 | 0.1 |
| DOCUSATE Na | — | — | — | — | 0.05 |
| TiO₂ | — | — | — | — | — |
| Physical | White gel/ | White gel/ | White gel/ | White gel/ | White gel/ |
| Appearance | translucent, | translucent, | translucent, | translucent, | translucent, |
| | shiny, | shiny, smooth, | shiny, smooth, | shiny, smooth, | shiny, smooth, |
| | smooth, thick | thick | thick | thick | thick |
| T3M 4° C./ | RAS/RAS/ | RAS/RAS/ | RAS/RAS/ | RAS/RAS/ | RAS/RAS/ |
| TA/30° C./40° C. | off-white gel/ | off-white gel/ | off-white gel/ | very light beige | very light |
| | light beige gel | light beige gel | light beige gel | gel/beige- | beige gel/ |
| | | | | orange gel | beige-orange |
| | | | | | gel |
| pH J + 1 | 4.79 | 4.86 | 4.92 | 4.70 | 4.74 |
| T3M/TA/30° C./ | 3.80/3.48/ | 3.89/3.70/ | 3.80/3.50/2.94 | 4.29/3.70/3.27 | 4.14/3.84/3.37 |
| 40° C. | 3.08 | 3.04 | | | |
| RV Viscosity | 129000 cPs | 122000 cPs | 131000 cPs | 104000 cPs | 85800 cPs |
| Needle 06, | (64.6%) | (60.9%) | (65.2%) | (51.8%) | (42.4%) |
| Speed = 5 rpm | 115000/ | 124000/ | 123000/ | 100000/ | 75000/ |
| T0 | 113000/ | 118000/ | 119000/ | 102000/ | 734000/ |
| T3M TA/30° C./ | 86800 | 97000 | 104000 | 88000 | 79200 |
| 40° C. | | | | | |
| T6M/TA/30° C./ | | | | | |
| 40° C. | | | | | |

TABLE 25

| | | | | | | |
|---|---|---|---|---|---|---|
| T1M/T4° C. | T0 (% LC) | 98.80 | 99.30 | 99.90 | 96.30 | 100.80 |
| T1M | T1M | | | | | |
| | 40° C. | 92.70 | 91.00 | 92.80 | 92.10 | 93.20 |
| | (% LC) | | | | | |
| | % T0 | 93.83 | 91.64 | 92.89 | 95.64 | 92.46 |
| T2M | T2M | | | | | |
| | TA | 103.60 | 104.00 | 104.70 | 103.40 | 105.40 |
| | (% LC) | | | | | |
| | % T0 | 104.86 | 104.73 | 104.80 | 107.37 | 104.56 |
| | T2M | | | | | |
| | 40° C. | 85.70 | 81.20 | 83.20 | 85.30 | 85.20 |
| | (% LC) | | | | | |
| | % T0 | 86.74 | 81.77 | 83.28 | 88.58 | 84.52 |
| T3M | T3M | | | | | |
| | TA | 97.70 | 97.20 | 98.80 | 97.90 | 98.80 |
| | (% LC) | | | | | |
| | % T0 | 98.89 | 97.89 | 98.90 | 101.66 | 98.02 |
| | T3M | | | | | |
| | 30° C. | 93.00 | 92.50 | 94.80 | 94.10 | 94.10 |
| | (% LC) | | | | | |
| | % T0 | 94.13 | 93.15 | 94.89 | 0.98 | 0.93 |
| | T3M | | | | | |
| | 40° C. | 67.00 | 56.20 | 60.00 | 65.20 | 65.50 |
| | (% LC) | | | | | |
| | % T0 | 67.81 | 56.60 | 60.06 | 67.71 | 64.98 |

TABLE 26
Series with TiO₂

| Composition/ | 0347.1149/ | 0347.1156/ | 0347.1180/ | 0347.1182/ | 0347.1183/ |
|---|---|---|---|---|---|
| Formula No. | 15.02479 | 15.02485 | 15.02487 | 15.02490 | 15.02506 |
| Manufacture Date | Dec. 7 2015 | Dec. 7 2015 | Dec. 7 2015 | Dec. 7 2015 | Dec. 9 2015 |
| INGREDIENT | % w/w | | | | |
| PURIFIED | QSP100 | QSP100 | QSP100 | QSP100 | QSP100 |
| WATER | | | | | |
| CD08467 | 1 | 1 | 1 | 1 | 1 |
| SIMULGEL ™ | 4 | 4 | 4 | 4 | 4 |
| 600PHA | | | | | |
| PHENOXETOL ™ | 0.8 | 0.8 | 0.8 | 0.8 | 0.8 |
| KOLLISOLV ® | — | 0.2 | 0.2 | 0.2 | 0.2 |
| P124 | | | | | |
| PROPANEDIOL- | — | 4 | 4 | 4 | 4 |
| 1,2 | | | | | |
| GLYCERIN 4810 | — | — | 4 | 4 | 4 |
| TITRIPLEX ® III | — | — | — | 0.1 | 0. 1 |
| SODIUM | — | — | — | — | 0.05 |
| DOCUSATE | | | | | |
| TiO₂ | 0.4 | 0.4 | 0.4 | 0.4 | 0.4 |
| Physical | White gel, | White gel, | White gel, | White gel, | White gel, |
| Appearance | shiny, smooth, | shiny, | shiny, smooth, | shiny, smooth, | shiny, smooth, |
| | thick | smooth, thick | thick | thick | thick |
| T3M 4° C./ | RAS/RAS/ | RAS/RAS/ | RAS/RAS/ | RAS/RAS/off- | RAS/RAS/ |
| TA/30° C./40° C. | white + fluid | RAS/light | RAS/light | white gel/light | off-white gel/ |
| | gel/light | beige gel | beige gel | beige gel | light beige gel |
| | beige + fluid | | | | |
| | gel | | | | |
| pH T0 | 4.70 | 4.72 | 4.75 | 4.71 | 4.83 |
| T3M 4° C./ | 3.83/3.53/3.02 | 3.74/3.45/ | 3.97/3.51/ | 4.23/3.89/3.38 | 4.22/3.89/3.25 |
| TA/30° C./40° C. | | 3.03 | 3.03 | | |
| Viscosity | 117000 cPs | 119000 cPs | 124000 cPs | 102000 cPs | 82000 cPs |
| RV, Needle 06, | (58.7%) | (60%) | (62%) | (50.5%) | (41%) |
| Speed = 5 rpm | | | | | |
| T0 | | | | | |
| T3M TA/30° C./ | 110000/ | 117000/ | 125000/ | 117000/ | 85000/ |
| 40° C. | 100000/ | 112000/ | 119000/ | 112000/ | 83000/ |
| | 32600 | 83200 | 91200 | 83200 | 81600 |

TABLE 27

| | | | | | | |
|---|---|---|---|---|---|---|
| T1M/T4° C. | T0 | 96.80 | 97.40 | 97.20 | 97.80 | 97.70 |
| | (% LC) | | | | | |
| T1M | T1M | | | | | |
| | 40° C. | 88.40 | 88.60 | 87.80 | 88.70 | 89.10 |
| | (% LC) | | | | | |
| | % T0 | 91.32 | 90.97 | 90.33 | 90.70 | 91.20 |
| T2M | T2M | | | | | |
| | TA | 101.00 | 102.50 | 102.00 | 103.20 | 103.20 |
| | (% LC) | | | | | |
| | % T0 | 104.34 | 105.89 | 105.37 | 106.61 | 106.61 |
| | T2M | | | | | |
| | 40° C. | 81.70 | 78.70 | 78.10 | 81.90 | 82.50 |
| | (% LC) | | | | | |
| | % T0 | 84.40 | 81.30 | 80.35 | 83.74 | 84.44 |
| T3M | T3M | | | | | |
| | TA | 96.60 | 98.20 | 97.40 | 97.60 | 98.00 |
| | (% LC) | | | | | |
| | % T0 | 99.79 | 100.82 | 100.21 | 99.80 | 100.31 |
| | T3M | | | | | |
| | 30° C. | 92.20 | 93.40 | 92.90 | 93.10 | 93.50 |
| | (% LC) | | | | | |
| | % T0 | 95.25 | 95.89 | 95.58 | 95.19 | 95.70 |
| | T3M | | | | | |
| | 40° C. | 62.20 | 51.30 | 51.70 | 60.70 | 63.10 |
| | (% LC) | | | | | |
| | % T0 | 64.26 | 52.67 | 53.19 | 62.07 | 64.59 |

From these studies, TiO2 contributes to the effective masking of coloration regardless of the gel composition; however, TiO2 destabilizes the formulation. The addition of propylene glycol and Poloxamer 124 are required for maintaining appropriate viscosity over time. Further studies have also shown that propylene glycol and Poloxamer 124 are required for the dispersion of Isopropylcarbonate Benzoyl Peroxide. Formulations containing propylene glycol and Poloxamer 124 are less chemically stable. The addition of a sequestering agent, such as disodium EDTA, appears to counter the chemically instability of the formulations containing propylene glycol and Poloxamer 124.

### Example 10: Preparation of Isopropylcarbonate Benzoyl Peroxide Formulations

The following Isopropylcarbonate Benzoyl Peroxide formulation was prepared as outlined in Process 1.

TABLE 28

| Composition/Formula No. | 0347.1198 |
|---|---|
| INGREDIENT | % w/w |
| PURIFIED WATER | QSP100 |
| CD08467 (GMP BATCH) | 5 |
| SIMULGEL ™ 600 PHA | 4 |
| PHENOXETOL ™ | 0.8 |
| KOLLISOLV ® P124 | 0.2 |
| PROPANEDIOL-1,2 | 4 |
| GLYCERIN 4810 | 4 |
| TITRIPLEX ® III | 0. 1 |
| SODIUM DOCUSATE | 0.05 |
| TITANIUM DIOXIDE 300309 | 0.4 |

TABLE 29
Process 1

| Composition/Formula No. | Phase | 0347.1198 |
|---|---|---|
| PURIFIED WATER | B | QSP100 |
| TITRIPLEX ® III | | 0.1 |
| SODIUM DOCUSATE | | 0.05 |
| GLYCERIN 4810 | | 4 |
| PURIFIED WATER | A | 20 |
| CD08467 (GMP BATCH) | | 5 |
| TITANIUM DIOXIDE 300309 | | 0.4 |
| KOLLISOLV ® P124 | | 0.2 |
| PROPANEDIOL-1,2 | | 4 |
| PURIFIED WATER | | 10 |
| PHENOXETOL ™ | | 0.8 |
| SIMULGEL ™ 600PHA | | 4 |

PHASE A:
In a beaker, weight the water + CD08467 + TiO₂ + KOLLISOLV ® P124 + PG. Mix by stirring in Silverson for 15 minutes at maximum speed.
PHASE B:
In a beaker, weigh the water + TITRIPLEXO III + Sodium Ducosate + Glycerin. Dissolve all while stirring.
While stirring, add Phase A to Phase B.
While stirring, add rinsing water.
While stirring, add the Phenoxyethanol then the SIMULGEL ™ 600

The Isopropylcarbonate Benzoyl Peroxide formulation prepared from Process I led to a very fine dispersion of TiO2 and Isopropylcarbonate Benzoyl Peroxide. However, the dispersion phase was difficult to implement further. Stirring with the Silverson machine provided a thickened mixture that took on the appearance of shaving cream.

Process 2 was then explored. The objective of Process 2 was to develop a manufacturing process that resulted in a more easily dispersible active phase by:
Reducing the quantity of water in this active phase,
Adding the sodium ducosate in this active phase in order to increase wetting ability, and
By introducing the TiO2 during the principle phase at the beginning of the process.

TABLE 30
Process 2

| Composition/Formula No. | Phase | 0347.1198 |
|---|---|---|
| PURIFIED WATER | B | QSP100 |
| TITRIPLEX ® III | | 0.1 |
| TITANIUM DIOXIDE 300309 | | 0.4 |
| GLYCERIN 4810 | | 4 |
| PURIFIED WATER | A | 10 |
| CD08467 (GAP BATCH) | | 5 |
| SODIUM DOCUSATE | | 0.05 |
| KOLLISOLV ® P124 | | 0.2 |
| PROPANEDIOL-1,2 | | 4 |
| PURIFIED WATER | | 15 |
| PHENOXETOL ™ | | 0.8 |
| SIMULGEL ™ 600PHA | | 4 |

PHASE A:
In a beaker, weight the water + Sodium Docusate + KOLLISOLV ® P124 + PG. Dissolve the Sodium Docusate while stirring, then add the CD08467. Mix by Silverson stirring for 15 minutes at maximum speed.
PHASE B:
In a beaker, weigh the water + TITRIPLEX ® III + TiO₂ + Glycerin. Dissolve all while stirring.
While stirring, add Phase A to Phase B.
While stirring, add rinsing water.
While stirring, add the Phenoxyethanol then the SIMULGEL ™ 600

Process 2 provided a very fine dispersion of the TiO₂ directly into the water of the principal phase. The dispersion phase is easy to implement. With stirring by a Silverson machine, the mixture remains fluid and homogeneous. However, care is required to the foam generated due to the presence of the docusate sodium in this phase.

A process compatible with preparation in a Magicplan reactor was then developed. Several problems for developing this process were encountered:
Silverson dispersion is not effective because this phase lacks wetting agents.
The final product is very bubbly. According to the order of the addition of raw materials, this air is incorporated either through the dispersion phase, or in the principal tank.

TABLE 30
Magic-plan Process

| Composition/Formula No. | | 0347.1198 |
|---|---|---|
| Ingredient | Phase | % w/w |
| PURIFIED WATER | B | QSP100 |
| TITRIPLEX ® III | | 0.1 |
| TITANIUM DIOXIDE 300309 | | 0.4 |
| SIMULGELTm 600PHA | | 1 |
| PURIFIED WATER | A | 10 |
| CD08467 (GAP BATCH) | | 5 |
| GLYCERIN 4810 | | 4 |
| KOLLISOLV ® P124 | | 0.2 |
| PROPANEDIOL-1,2 | | 4 |
| PURIFIED WATER | | 15 |
| SODIUM DOCUSATE | C | 0.05 |
| PURIFIED WATER | | 5 |
| PHENOXETOL ™ | | 0.8 |
| SIMULGEL ™ 600PHA | | 3 |

The following aspects are featured in the Magic-plan process:
In this process, the TiO₂ is incorporated into the principal tank at the beginning of manufacturing.
A fraction of SIMULGEL™ 600 is added at the beginning of manufacturing in order to increase shearing and avoid the formation of foam.
Glycerin is added at the dispersion phase in order to result in better wetting of the Isopropylcarbonate Benzoyl Peroxide.
Docusate is added to the formula dissolved in water at the end of the process in order to avoid the formation of foam.

An optional step for the Magic-plan process is the reduce the crystal size of the Isopropylcarbonate Benzoyl Peroxide by using a colloid mill. An exemplary process of dispersion using a colloid mill to reduce Isopropylcarbonate Benzoyl Peroxide used the following dispersion phase (about 500 g):

TABLE 31

| INGREDIENT | % w/w |
|---|---|
| PURIFIED WATER | 10 |
| GLYCERIN | 4 |
| KOLLISOLV ® P124 | 0.2 |
| PROPANEDIOL 1,2 | 4 |
| CD08467 | 5 |

The above dispersion phase was stirred in the Magiclab tank that was coupled with the colloid mill. During this experiment, the speed of the mill, the milling time, the size of the mill, and air gap were varied. Fractions sampled during the different milling stages were observed under the microscope and the following observations were made as noted in the below table.

| | | | | Active | |
|---|---|---|---|---|---|
| | | | | phase | |
| | | Milling | | T° C. | |
| | Speed | time | Air gap | during | |
| No. | (Tr/min) | (minutes) | (μm) | milling | Notes |
| 1 | 4000 | 5 min | 5000 (max) | 18° C. | |
| 2 | 5200 | 5 min | 2500 | 17° C. | |
| 3 | 13400 | 5 min | 160 (mini) | from 28° C. | Ice cubes + |
| | | | | to 34° C. | double |
| | | | | | envelope |
| 4 | 13400 | 10 min | 160 (mini) | 28° C. | Ice cubes + |
| | | | | | double |
| | | | | | envelope |
| 5 | 20000 | 2 min * | 160 (mini) | 40° C. | * Stirring |
| | | | | | halted due |
| | | | | | to an increase |
| | | | | | in the |
| | | | | | T° C. |

The samples observed under microscope, and the size of the crystals was measured. In this experiment, the desired size of 1 to 60 μm was obtained after 15 min at 13400 tr/min and the smaller air gap (160 μm). A higher speed (20000 tr/min) resulted in a decrease in the size of the particles but the temperature is very difficult to control and increases very quickly. The use of the Magic-lab connected to the colloid mill resulted in the reduction in the size of CD08467 particles as a function of the air gap, speed and stirring time.

The following table shows an exemplary conditions for manufacturing the Isopropylcarbonate Benzoyl Peroxide formulation.

TABLE 33

| | PROCEDURE | PARAMETERS | COMMENTS |
|---|---|---|---|
| STEP 1: MAIN | TITRIPLEX SOLUBILISATION | T° C.: TA | Appearance of |
| PHASE | In the main tank, charge water and add | Speed: 40 rpm | the preparation |
| | TITRIPLEX ® III under homogenization (low | Emulsor: 4000 rpm | at the end of |
| | speed) | Time: 15 min | the step: |
| | Control step - IPC | | Transparent |
| | Visual control, Transparent solution | | solution |
| STEP 2: | ACTIVE PHASE - CD08467 | T° C.: TA | Appearance of |
| ACTIVE PHASE | DISAGGREGATION | Stirrer/Homogenizer: | the preparation |
| DISPERSION | Weigh in a beaker CD08467, WATER, | Silverson | at the end of |
| CD08467 | PROPYLENE GLYCOL, GLYCERINE and | Speed: 9000 rpm | the step: |
| | KOLLISOLV ® P124 | Time: 10 min | White liquid |
| | Place the beaker in an ice bath during the | | phase (with |
| | disaggregation step. | | some foam |
| | Increase the speed slowly until 9000 rpm to | | residue) |
| | minimize foam formation | | |
| STEP 3: | HOMOGENIZATION OF TITANIUM | T° C.: TA | Appearance of |
| TITANIUM | DIOXIDE | Speed: 40 rpm | the preparation |
| DIOXIDE | Add slowly TITANIUM DIOXIDE the main | Emulsor: 4000 rpm | at the end of |
| HOMOGENI- | tank | Time: 10 min | the step: |
| ZATION | Control step - IPC | | White liquid |
| | Visual inspection of White liquid mixture | | phase |
| STEP 4: | DISPERSION OF SIMULGEL ™ 600 PHA | T° C.: TA | Appearance of |
| SIMULGEL | IN THE MAIN PHASE | Speed: 70 rpm | the preparation |
| PART 1 DIS- | Add the first part of SIMULGEL ™ 600(1%) | Emulsor: 9000 rpm | at the end of |
| PERSION | in the main tank | Time: 10 min | the step: |
| | Control step | | Fluid White |
| | Visually confirm that the white liquid phase is | | phase |
| | completely homogeneous and free from | | |
| | SIMULGEL ™ 600 agglomerates | | |
| STEP 5: | ACTIVE PHASE HOMOGENEISATION IN | ° C.: TA | Appearance of |
| ACTIVE PHASE | MAIN PHASE | Speed: 70 rpm | the preparation |
| HOMOGENI- | Transfer the active phase containing | Emulsor: 9000 rpm | at the end of |
| ZATION IN | CD08467 | Time: 5 min | the step: |
| MAIN PHASE | from step 2 into the main phase | | Fluid White |
| | Rinse all vessels and agitator used for the | | phase |
| | dispersion with rinse water | | |
| | Add rinse water in the main phase | | |
| STEP 6: | Add PHENOXETHOL ™ in the main Phase | ° C.: TA | Appearance of |
| DISPERSION | | Speed: 70 rpm | the preparation |
| OF PHEN- | | Emulsor: 9000 rpm | at the end of |
| OXETHOL | | Time: 5 min | the step: |
| | | | Fluid White |
| | | | phase |
| STEP 7: | In a Beaker, under magnetic stirring, dissolve | T° C.: 40° C. | Appearance of |
| SOLUBILISA- | SODIUM DOCUSATE in WATER | Magnetic stirring | the preparation |
| TION OF SODI- | Control step | Speed: 400 rpm | at the end of |
| UM | Visually confirm that SODIUM DOCUSATE | Time: 35 min | the step: |
| DOCUSATE | is completely dissolved | | Clear solution |
| STEP 8: | Slowly transfer the SODIUM DOCUSATE | ° C.: TA | Appearance of |
| HOMOGENIZA- | annex phase into the Main phase | Speed: 70 rpm | the preparation |
| TION OF THE | Mix using gentle agitation to avoid foam | Emulsor: 900 rpm | at the end of |
| SODIUM | formation | Time: 5 min | the step: |
| DOCUSATE | Avoid foam formation by using gentle | | Fluid White |
| ANNEX PHASE | agitation | | phase |
| STEP 9: | Add the part II of SIMULGEL ™ 600PHA in | ° C.: TA | Aspect |
| SIMULGEL | the main Phase | Speed: 120 rpm | preparation at |
| SECOND | Increase stirring speed slowly to prevent the | Emulsor: 20000 rpm | the end of the |
| PHASE | formation of foam | Time: 30 min | step: |
| DISPERSION | Control step | | Thick white gel |
| | Visually confirm that the thick white gel is | | |
| | completely homogeneous and free from | | |
| | SIMULGEL ™ 600 agglomerates | | |

### Example 11: Stability Studies of Exemplary Isopropylcarbonate Benzoyl Peroxide Formulations

The following tables show exemplary Isopropylcarbonate Benzoyl Peroxide formulations and corresponding stability studies.

TABLE 34
3% Isopropylcarbonate Benzoyl Peroxide Gel [0347.1199]

| Article description | INCI Name | Function | % |
|---|---|---|---|
| CD068467 | ISOPROPYLCARBONATE BENZOYL PEROXIDE | Active Ingredient | 3% |
| TIO₂ SENSIENT 300309 PHARMA | TITANIUM DIOXIDE | Opacifier | 0.4% |
| DOCUSTA GER | SODIUM DOCUSTE | Surfactant | 0.05 |
| SODIUM PH EU | | | |
| EAU PURIFEE | | Vehicle | 83.45 |
| GLYCERIN 4810 | GLYCEROL | Humectant | 4 |
| PLANT | | | |
| KOLLISOLVO ® P124 | POLOXAMER 124 | Liquid Wetting | 0.2 |
| | | Surfactant | |
| SIMULGEL ™ 600 PHARMA | ACRYLAMIDE/SODIUM | Gelling Agent | 4 |
| | ACRYLOYLDIMETHYL | | |
| | TAURATE COPOLYMER/ | | |
| | ISOHEXADECANE/ | | |
| | POLYSORBATE 80 | | |
| PHENOXETOL ™ | PHENOXYETHANOL | Preserving Agent | 0.8 |
| PROPANEDIOL-1,2 | PROPYLENE GLYCOL | Pro-penetrating Agent | 4 |
| TITRIPLEX ® III | DISODIUM EDTA | Sequestering Agent | 0.1 |

TABLE 35
3% Isopropylcarbonate Benzoyl Peroxide Gel [0347.1216]

| Article description | INCI Name | Function | % |
|---|---|---|---|
| CD068467 | ISOPROPYLCARBONATE | Active | 3% |
| | BENZOYL PEROXIDE | Ingredient | |
| HOMBITANT | TITANIUM DIOXIDE | Opacifier | 0.4% |
| TITANIUM DIOXIDE | | | |
| FF PHARMA | | | |
| DOCUSTA GER | SODIUM DOCUSTE | Surfactant | 0.05 |
| SODIUM PH EU | | | |
| EAU PURIFEE | | Vehicle | 83.75 |
| GLYCERIN 4810 | GLYCEROL | Humectant | 4 |
| PLANT | | | |
| KOLLISOLV ® | POLOXAMER 124 | Liquid | 0.2 |
| P124 | | Wetting | |
| | | Surfactant | |
| COS SIMULGEL ™ | ACRYLAMIDE/SODIUM | Gelling | 4 |
| 600 | ACRYLOYLDIMETHYL | Agent | |
| | TAURATE COPOLYMER/ | | |
| | ISOHEXADECANE/ | | |
| | POLYSORBATE 80 | | |
| PHENOXETOL ™ | PHENOXYETHANOL | Preserving | 0.4 |
| | | Agent | |
| PROPANEDIOL-1,2 | PROPYLENE GLYCOL | Pro- | 4 |
| | | penetrating | |
| | | Agent | |
| TITRIPLEX ® III | DISODIUM EDTA | Sequestering | 0.2 |
| | | Agent | |

TABLE 36
3% Isopropylcarbonate Benzoyl Peroxide Gel [0347.1217]

| Article description | INCI Name | Function | % |
|---|---|---|---|
| CD08467 | ISOPROPYLCARBONATE | Active Ingredient | 3% |
| | BENZOYL PEROXIDE | | |
| COS EUROVIT | TITANIUM DIOXIDE | Opacifier | 0.4% |
| TITANIUM DIOXIDE | | | |
| 500095X25 | | | |
| DOCUSTA GER | DIETHYL SODIUM | Surfactant | 0.05 |
| SODIUM PH EU | SULFOSUCCINATE | | |
| EAU PURIFEE | WATER/AQUA | Vehicle | 83.75 |
| GLYCERIN 4810 | GLYCEROL | Humectant | 4 |
| PLANT | | | |
| KOLLISOLV ® P124 | POLOXAMER 124 | Liquid Wetting Surfactant | 0.2 |
| COS SIMULGEL ™ 600 | ACRYLAMIDE/SODIUM | Gelling Agent | 4 |
| | ACRYLOYLDIMETHYL | | |
| | TAURATE COPOLYMER/ | | |
| | ISOHEXADECANE/ | | |
| | POLYSORBATE 80 | | |
| PHENOXETOL ™ | PHENOXYETHANOL | Preserving Agent | 0.4 |
| PROPANEDIOL-1,2 | PROPYLENE GLYCOL | Pro-penetrating Agent | 4 |
| TITRIPLEX ® III | DISODIUM EDTA | Sequestering Agent | 0.2 |

TABLE 37

| | | 0347.1199/17.00721: CD08467 3% GEL | | | | |
|---|---|---|---|---|---|---|
| | | Packaged in 30 g polifoïl PP000087 tube | | | | |
| | | SIMULGEL ™ 600 Pharma, TiO₂ SENSIENT 300309 pharma, Phenoxyethanol 0.8%, | | | | |
| | | EDTA 0.1% | | | | |
| | Stability studies done at: | Sophia | | | | Alby |
| | | T0 | T1 | T3 | T6 | T12 |
| Macroscopic appearance | TA | White gel | NA | NA | Off-white | Beige gel |
| Non pourable white to | | | | | gel | |
| beige opaque gel | 30° C. | — | Slightly off- | Off-white gel | Slightly | Slightly yellow gel |
| | | | white gel | | yellow gel | |
| | 5° C. | — | — | White gel | White gel | |
| | Cycles 5° C./40° C. | — | — | Off-white gel | — | — |
| | Cycles −20° C./+25° C. | — | — | Off-white gel | — | — |
| Microscopic appearance | TA | 100% < 100 μm | 100% < 100 μm | 99.997 < 100 μm | — | 100% < 100 μm |
| Particle size 99% < 100 μm | 30° C. | — | 100% < 100 μm | 99.997 < 100 μm | — | 100% < 100 μm |
| | Cycles 5° C./40° C. | — | — | 99.997 < 100 μm | — | — |
| | Cycles −20° C./+25° C. | — | — | 99.997 < 100 μm | — | — |
| pH | TA | 4.2 | 4.0 | 3.7 | 3.5 | 3.4 |
| 2.8-5.0 | 30° C. | — | 3.8 | 3.5 | 3.3 | 3.1 |
| | Cycles 5° C./40° C. | — | — | 3.6 | — | — |
| | Cycles −20° C./+25° C. | — | — | 3.7 | — | — |
| Viscosity | RT | 36491 mPa.s | 35298 mPa.s | 37031 mPa.s | 36386 mPa.s | — |
| Brookfield RVDVII + | | | | | | |
| Spindle 29, 14 rpm, 25° C. | 30° C. | — | 36354 mPa.s | 37233 mPa.s | 36610 mPa.s | — |
| Report results | Cycles 5° C./40° C. | — | — | 37265 mPa.s | — | — |
| | Cycles −20° C./+25° C. | — | — | 35667 mPa.s | — | — |
| Chemical stability | RT | 100.2% | 100.8% | 102.9% | 100.9% | 100.4% |
| Phenoxyethanol % LC | 30° C. | — | 102.6% | 103.0% | 101.0% | 99.7% |
| 90%-110% (Sophia) | Cycles 5° C./40° C. | — | — | 102.5% | — | — |
| 90%-120% (Alby) | Cycles −20° C./+25° C. | — | — | 101.6% | — | — |
| CD08467 % LC | RT | 100.7% | 101.7% | 100.2% | 99.3% | 95.6% |
| 90%-110% tbc | 30° C. | — | — | | — | 86.4% |
| | Cycles 5° C./40° C. | — | — | 99.2% | — | — |
| | Cycles −20° C./+25° C. | — | — | 99.6% | — | — |

| | |
|---|---|
| Microbiological stability | Meet the criteria of Ph Eur. Criteria A |

TABLE 38

| | | 0347.1216/17.00797: CD08467 3% GEL | | | | |
|---|---|---|---|---|---|---|
| | | Packaged in 30 g polifoïl PP000087 tube | | | | |
| | | SIMULGEL ™ 600 COS, TiO₂: Hombitant Titanium dioxide FF Pharma, | | | | |
| | | Phenoxyethanol 0.4%, EDTA 0.2% | | | | |
| | Stability studies done at: | Sophia | | | Alby | |
| | | T0 | T1 | T3 | T6 | T12 |
| Macroscopic appearance | RT | NR | | | Off-white gel | |
| Non pourable white to | 30° C. | | | | Slightly orange gel | |
| Microscopic appearance | RT | NR | | | 100% < 100 μm | |
| Particle size 99% < 100 μm | 30° C. | | | | 100%<100 μm | |
| pH | RT | NR | | | 3.8 | |
| 2.8-5.0 | 30° C. | | | | 3.5 | |
| Viscosity | RT | NR | | | | |
| Brookfield RVDVII+ | | | | | | |
| Spindle 29, 14 rpm, 25° C. | | | | | | |
| Report results | | | | | | |
| Chemical stability | RT | NR | | | 101.0% | |
| Phenoxyethanol % LC | 30° C. | | | | 101.5% | |
| 90%-110% (Sophia) | | | | | | |
| 90%-120% (Alby) | | | | | | |
| CD08467 % LC | RT | NR | | | 97.6% | |
| 90%-110% tbc | 30° C. | | | | 96.0% | |

TABLE 39

| | | 0347.1217/18.00007: CD08467 3% GEL | | | | |
|---|---|---|---|---|---|---|
| | | Packaged in 30 g polifoïl PP000087 tube | | | | |
| | | SIMULGEL ™ 600 COS, TiO₂: COS EUROVIT Titanium dioxide, | | | | |
| | | Phenoxyethanol 0.4%, EDTA 0.2% | | | | |
| | Stability studies done at: | Sophia | | Alby: | | |
| | | T0 | T1 | T3 | T6 | T12 |
| Macroscopic appearance | RT | NR | | White gel | On going Alby | |
| Non pourable white to beige | 30° C. | | | Off-white gel | On going Alby | |
| opaque gel | | | | | | |
| Microscopic appearance | RT | NR | | 100% < 100 μm | | |
| Particle size 99% < 100 μm | 30° C. | | | 100% < 100 μm | | |
| pH | RT | NR | | 4.0 | On going Alby | |
| 2.8-5.0 | 30° C. | | | 3.7 | On going Alby | |
| Viscosity | RT | NR | | | | |
| Brookfield RVDVII + Small | | | | | | |
| volume adapter 14 g; Spindle | | | | | | |
| 29; 14 rpm, 25° C. | | | | | | |
| Chemical stability | RT | NR | | 100.0% | On going Alby | |
| Phenoxyethanol % LC | 30° C. | | | 100.0% | On going Alby | |
| 90%-110% (Sophia); 90%- | | | | | | |
| 120% (Alby) | | | | | | |
| CD08467 | RT | 97.5% | | 95.6% | On going Alby | |
| % LC | 30° C. | | | 94.0% | On going Alby | |
| 90%-110% tbc | | | | | | |

### Example 12: Exemplary Isopropylcarbonate Benzoyl Peroxide Formulation

An exemplary Isopropylcarbonate Benzoyl Peroxide formulations is shown below. The composition in Table 40 is herein referred to as “Composition B.”

TABLE 40
3% Isopropylcarbonate Benzoyl Peroxide Gel (Composition B)

| Component | INCI Name | Function | % |
|---|---|---|---|
| CD068467 | ISOPROPYLCARBONAIL | Active | 3 |
| | BENZOYL | Ingredient | |
| | PEROXIDE | | |
| TITANIUM | TITANIUM DIOXIDE | Opacifier | 0.4 |
| DIOXIDE 300309 | | | |
| DOCUSATE | DIETHYLHEXYL | Surfactant | 0.05 |
| SODIUM | SODIUM | | |
| | SULFOSUCCINATE | | |
| EAU PURIFEE | WATER | Vehicle | 83.75 |
| | GLYCEROL | Humectant | 4 |
| KOLLISOLV ® P124 | POLOXAMER 124* | Liquid | 0.2 |
| | | Wetting | |
| | | Surfactant | |
| SIMULGEL ™ 600 | ACRYLAMIDE/SODIUM | Gelling | 4 |
| PHA** | ACRYLOYLDIMETHYL | Agent | |
| | TAURATE | | |
| | COPOLYMER/ | | |
| | ISOHEXADECANE/ | | |
| | POLYSORBATE 80 | | |
| PHENOXETOL ™ | PHENOXYETHANOL | Preserving | 0.4 |
| | | Agent | |
| PROPANEDIOL-1,2 | PROPYLENE GLYCOL | Pro- | 4 |
| | | penetrating | |
| | | Agent | |
| TITRIPLEX III | DISODIUM EDTA | Sequestering | 0.2 |
| | | Agent | |

*Contains tocopherol as an antioxidant (0.00001% to 0.00003% of final composition)
**May contain sorbitan oleate (0% to 0.2% of final composition)

### Example 13: Clinical Study

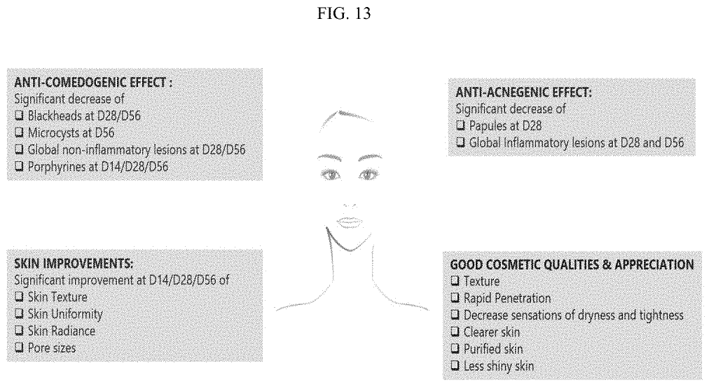

A clinical study of was conducted to evaluate cutaneous acceptability in people/subjects with mild to moderate facial acne vulgaris of Composition B. The clinical study also evaluated the effect of the composition on inflammation and lesions, efficacy (based on clinical scoring by a dermatologist managing the study) on spots, blackheads, porphyrin, and pores over time, the sebo-regulating effect, and qualitative and quantitative sebum composition. Overall, the composition was well-tolerated with many positive effects associated (FIG. 13).

### Subjects and Protocol

A group of 76 subjects were screened for 44 randomized subjects to receive the composition in a 56 day study (evaluation on days 0, 14, 28, and 56). The results were acquired based on at least 40 subjects that received the composition and applied the product once daily. The inclusion criteria of subjects were:
1. 17 Male and 27 Female subjects aged between 14 to 35 years old;
2. Skin phototype of II to IV of all ethnicities;
3. 20% greasy skin, 80% combination skin;
4. Panel of subject with combined and oily skin: sebumetric measurements at D0>120 μg/cm² for 20 volunteers;
5. Mild to moderate facial acne vulgaris defined by:
   a. Investigator's Global Assessment (IGA) score of 2 to 3 (mild to moderate acne).
   b. A minimum number of non-inflammatory lesions:10-30
   c. A minimum number of inflammatory lesions: 7-15
   d. A total number of lesions below 45.

### Efficacy

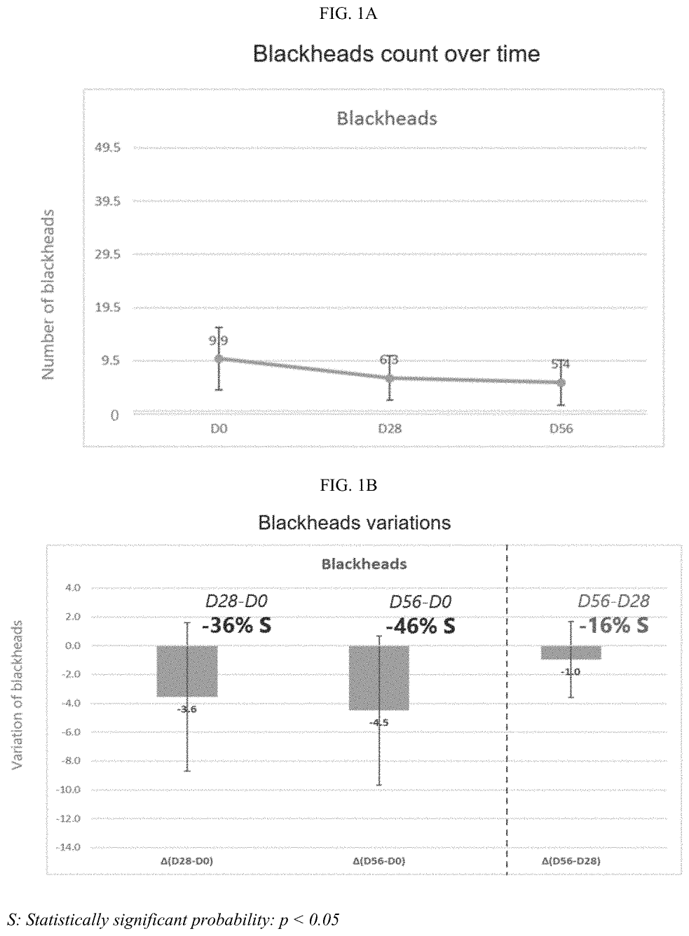

As illustrated in FIGS. 1-6, the composition provided significant improvements including the significant and rapid onset (1 month) of action and continuous improvement (2 months) of blackheads (FIGS. 1A and 1B). The number of conspicuous blackheads decreased (i.e., 10% at day 14, 10% at day 28, and 14% at day 56). Additionally, the area of conspicuous blackheads also decreased (i.e., 11% at day 14, 11% at day 28, and 15% at day 56).

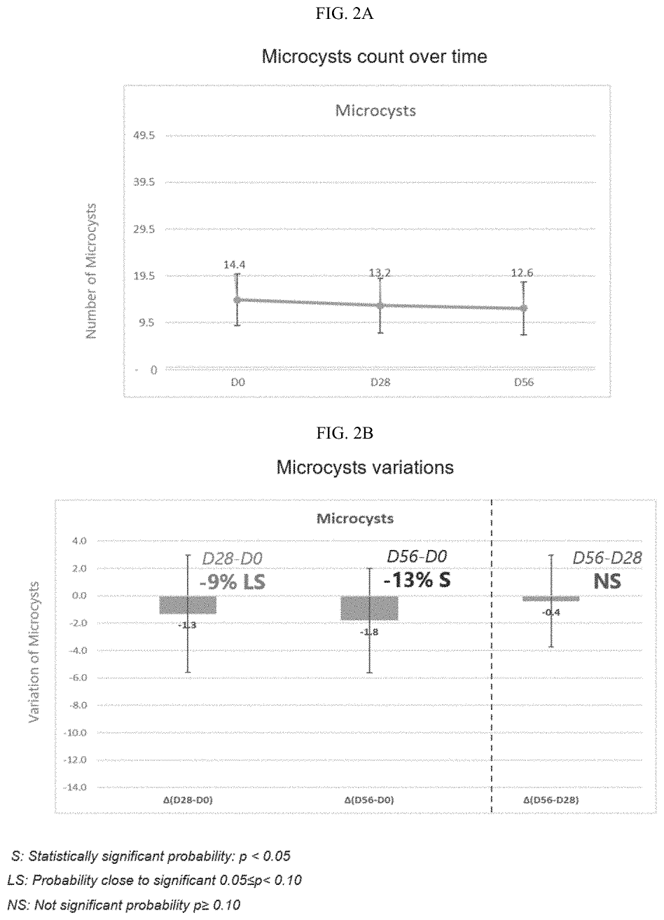

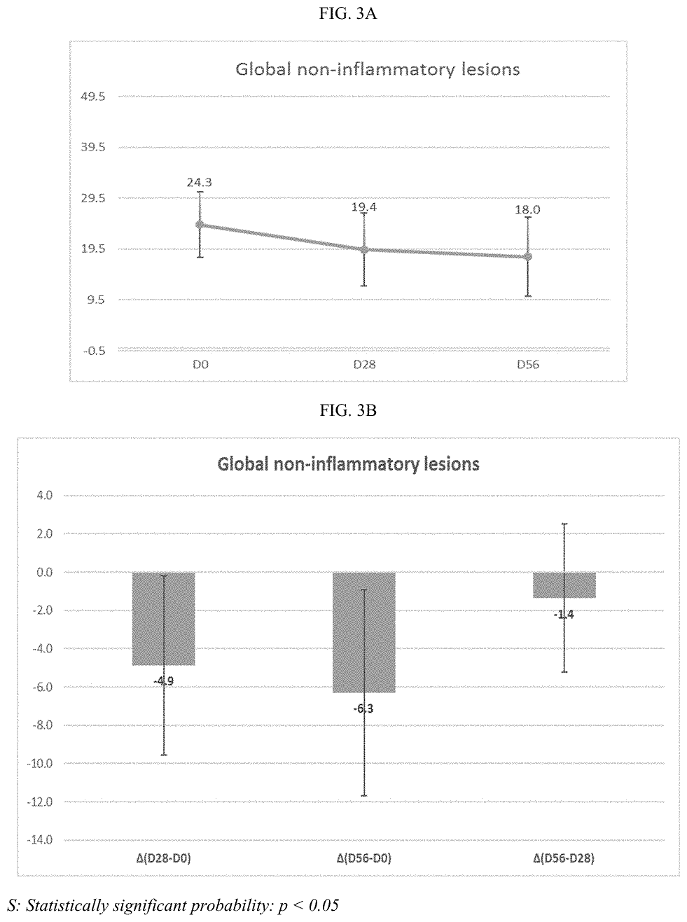

The composition also provided significant improvement of microcysts after 2 months (FIGS. 2A and 2B), significant and rapid onset (1 month) of action and continuous improvement (2 months) of non-inflammatory lesions (FIGS. 3A and 3B), and significant and rapid decrease (14 days) of P. acnes and maintained efficacy over the duration of the study (2 months). The number of porphyrins decreased (i.e., 43% at day 14, 45% at day 28, and 47% at day 56).

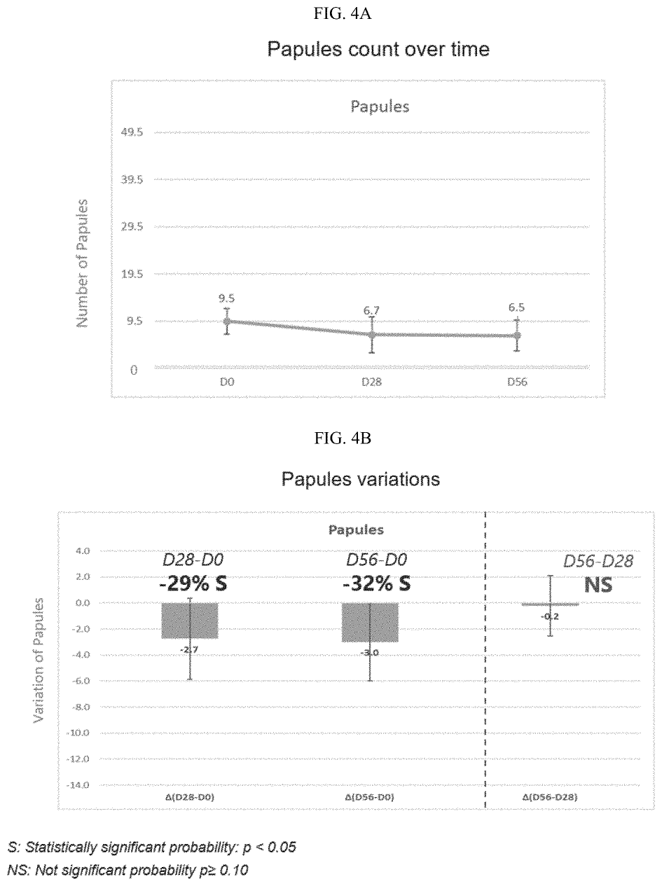

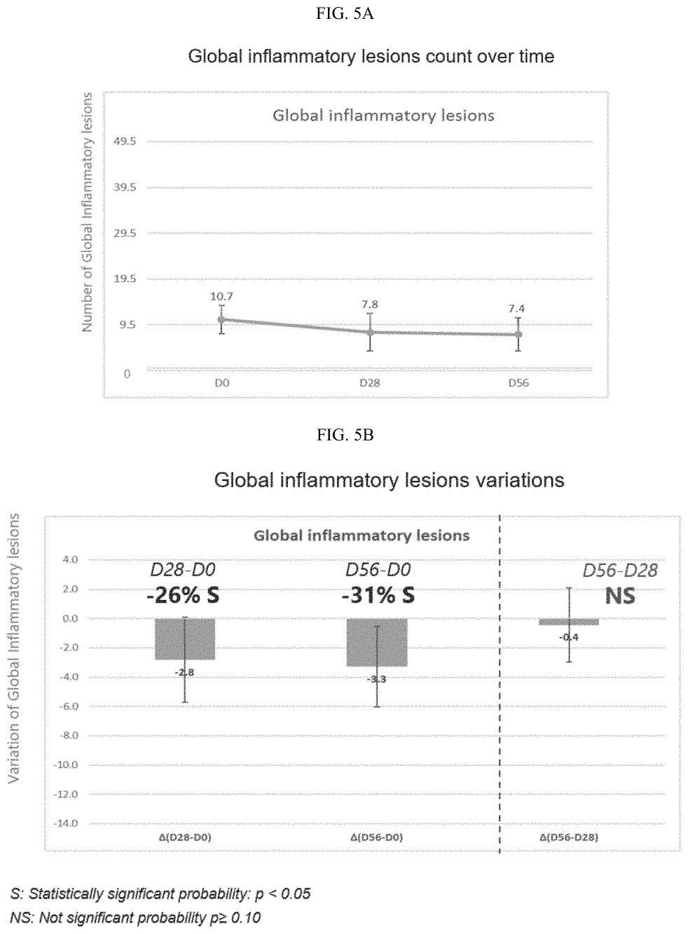

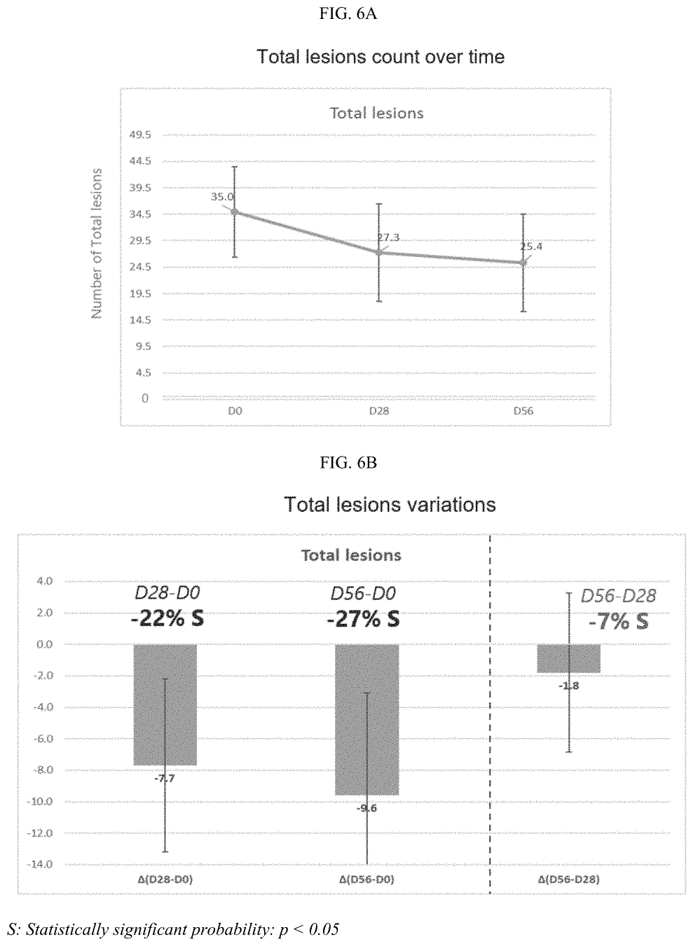

Additionally, the composition provided significant and rapid onset (1 month) of action decreasing papules and maintained efficacy over the duration of the study (2 months) (FIGS. 4A and 4B), significant and rapid onset (1 month) of action decreasing inflammatory lesions and maintained efficacy over the duration of the study (2 months) (FIGS. 5A and 5B), and significant and rapid onset (1 month) of action and continuous improvement (2 months) of lesions (non-comedogenic and non-acnegenic effect) (FIGS. 6A and 6B).

### Skin Improvements

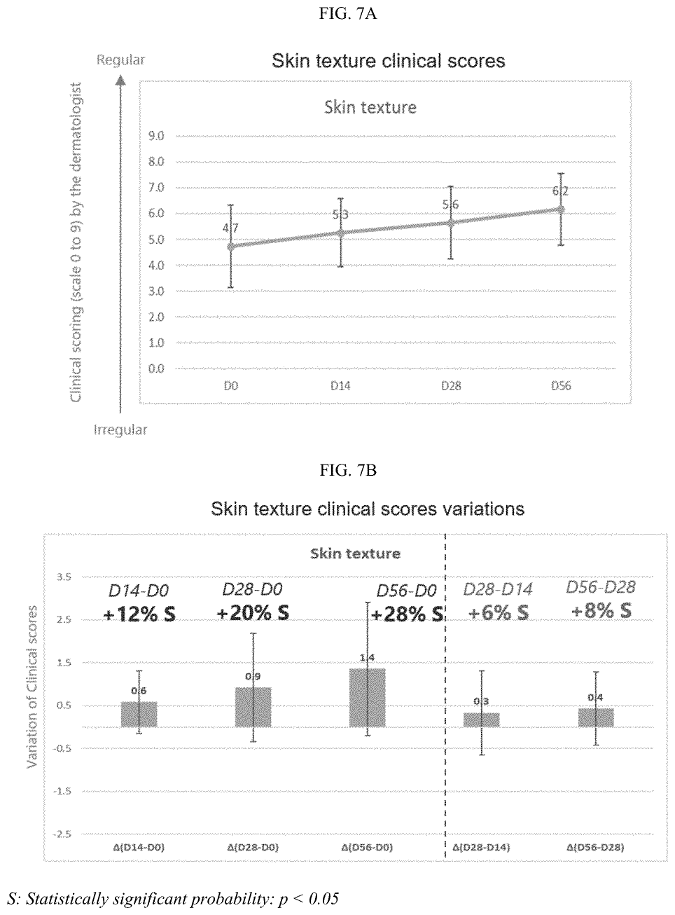

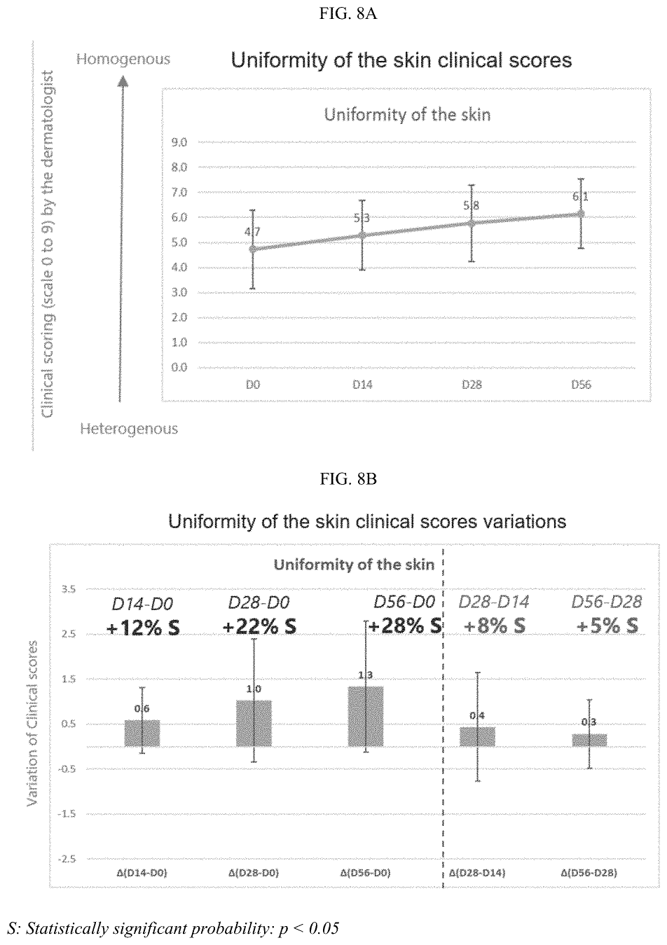

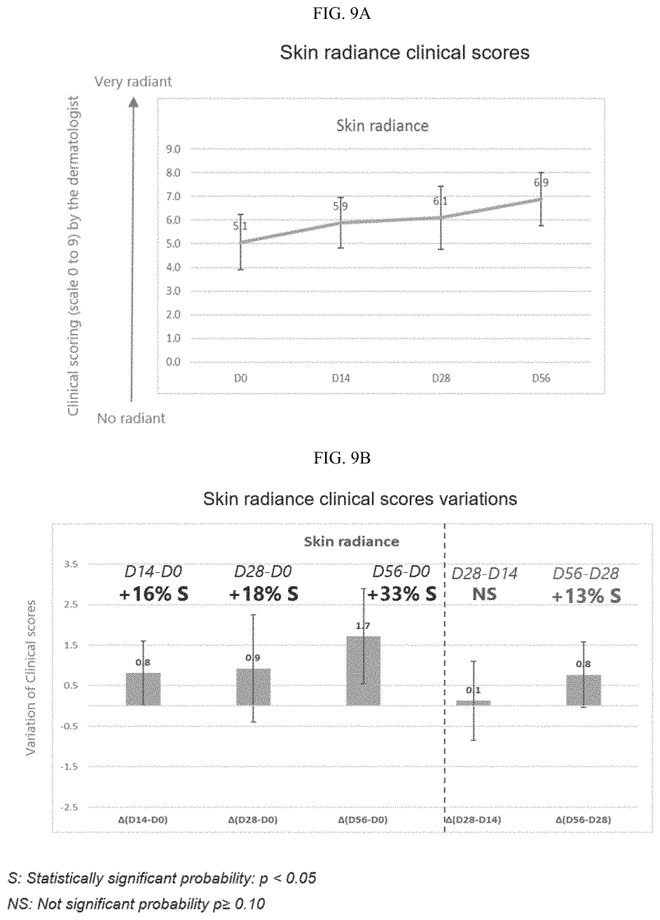

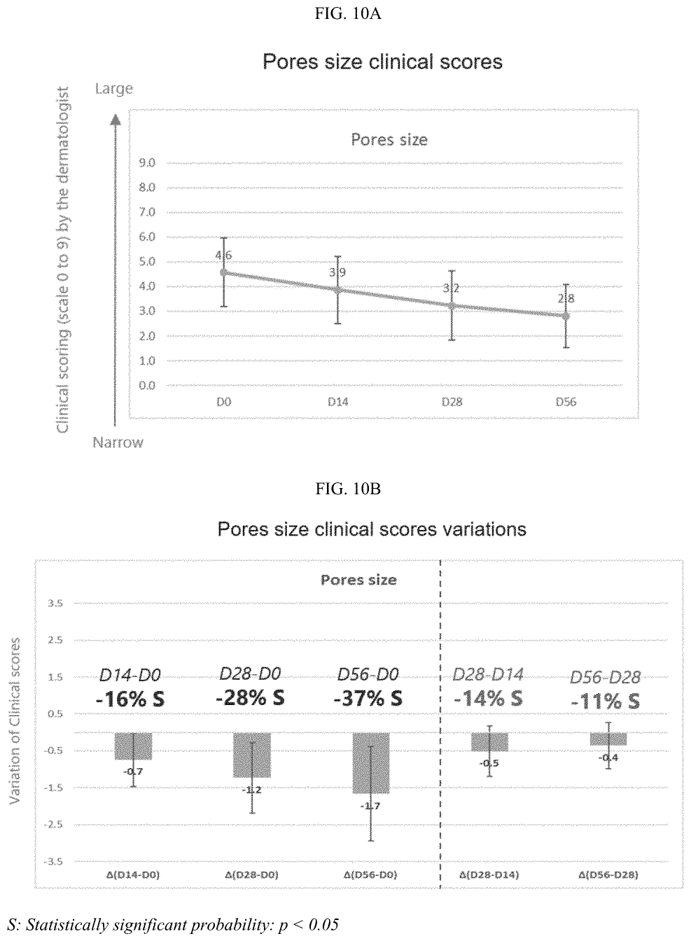

As illustrated in FIGS. 7A and 7B, the composition provided significant and rapid onset (14 days) of action and continuous improvement (1 & 2 months) of skin texture. It also provided significant and rapid onset (14 days) of action and continuous improvement (1 & 2 months) of skin uniformity (FIGS. 8A and 8B), significant and rapid onset (14 days) of action and continuous improvement (2 months) of skin radiance (FIGS. 9A and 9B), and significant and rapid onset (14 days) of action and continuous improvement (1 & 2 months) of skin pore size (FIGS. 10A and 10B). The number conspicuous of pores decreased (i.e., 15% at day 14 and 14% at day 56), the conspicuous volume of pores decreased (i.e., 21% at day 14 and 18% at day 56), and the conspicuous density of pores decreased (i.e., 13% at day 14, 14% at day 28, and 14% at day 56).

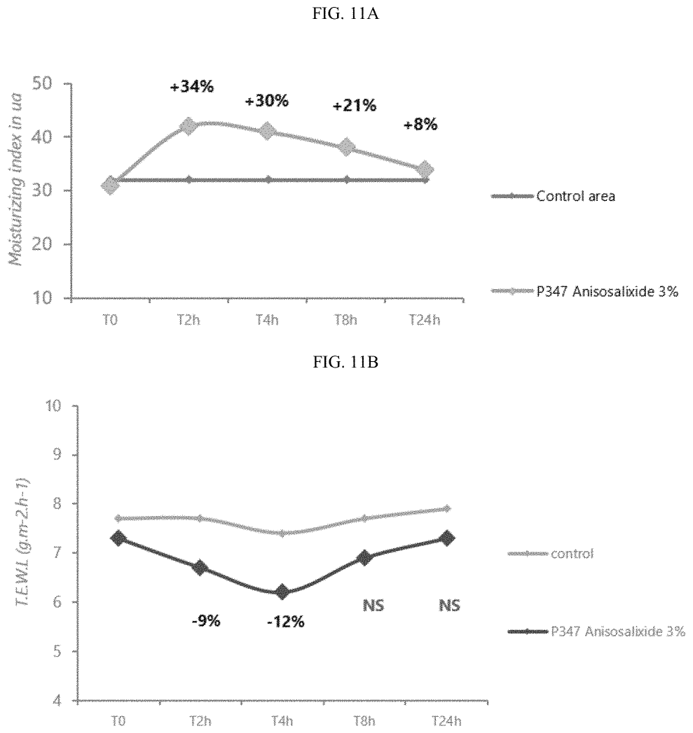

Additionally, the composition provided statistically significant moisturizing effect of epidermis superficial layers up to 24 h after 1 application (FIG. 11A) and significant decrease up to 4 h showing that it presents protective effect after 2 h and 4 h and respects and preserves the cutaneous barrier after 8 h and 24 h after 1 application (FIG. 11B).

### Sebum

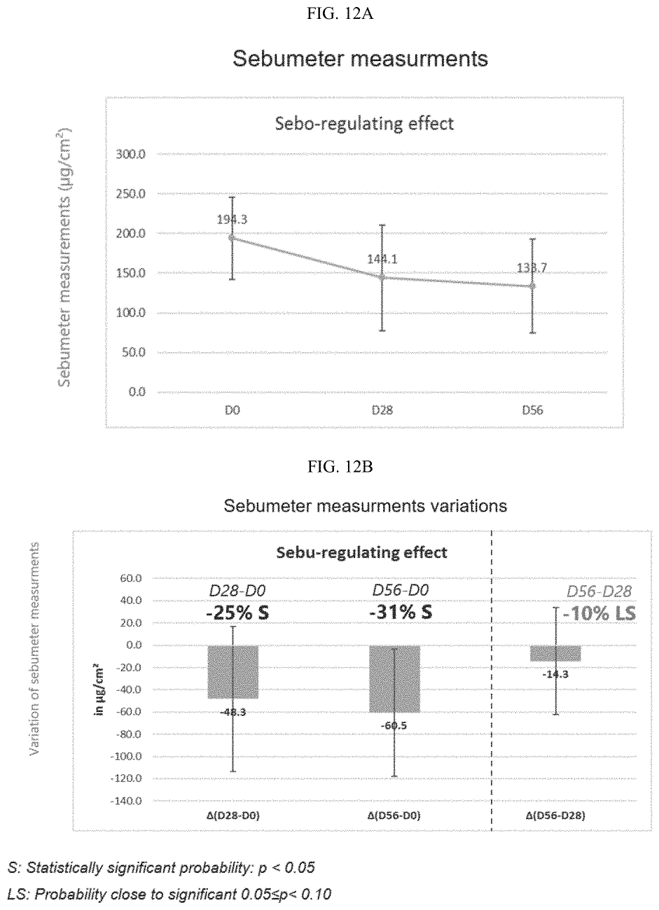

As illustrated in FIGS. 12A and 12B, the composition provided significant and rapid onset (1 month) of action and continuous improvement and maintained efficacy (2 months) of sebo-regulating effect. The composition provided a reduction of sebum production after 1 month and maintained efficacy (2 months). The quantitative analysis of the sebum (GC/MS method) is provided in Tables 41 and 42 demonstrating the improved quality.

TABLE 41
Sebum Analysis
Sebum quantative
analysis GC/MS method

| Biochemical Exploration | D28 vs D0 | | D56 vs D0 | |
|---|---|---|---|---|
| Extracted lipids (μg/mg) | −11% | S | −12% | S |
| Squalene (SQ) (μg/mg) | −17% | S | −16% | S |
| Waxes (% Area/mg) | −11% | S | −12% | S |

S: Statistically significant probablity: p < 0.05

[symbol]

[symbol]

indicates data missing or illegible when filed

TABLE 41
Sebum Analysis
Sebum quantative analysis
GC/MS method
and LC/MS for SQOOH

| Biochemical Exploration | D28 vs D0 | | D56 vs D0 | |
|---|---|---|---|---|
| Cholesterol (μg/mg) | 4% | S | 3% | S |
| TriGlycerides (% Area/mg) | 1% | NS | −2% | NS |
| Free Fatty Acids (% Area/mg) | −14% | S | −17% | S |
| Ratio TriGlycerides/FFA (%) | +18% | S | +20 | S |
| Peroxidized Squalene | −23% | S | −23% | S |
| (SQOOH) (ng/mg) | | | | |
| Ratio [SQOOH]/[SQ] | −9% | S | −9% | S |

S: Statistically significant probability: p < 0.05

[symbol]

[symbol]

indicates data missing or illegible when filed

What is claimed is:

1. A topically applicable composition comprising:
i) Isopropylcarbonate Benzoyl Peroxide;
ii) an opacifier; and
iii) a preserving agent.

2. The topically applicable composition of claim 1, wherein the composition comprises about 0.1% to about 10% by weight of Isopropylcarbonate Benzoyl Peroxide.

3. The topically applicable composition of claim 1, wherein the opacifier is bismuth oxychloride, titanium dioxide, fluorphlogopite, mica, iron oxide, nylon polymer, polymethyl methacrylate, boron nitride, kaolin, glycol distearate, styrene copolymer, a fatty alcohol, or any combination thereof.

4. The topically applicable composition of claim 1, wherein the composition comprises about 0.1% to about 2.5% by weight of the opacifier.

5. The topically applicable composition of claim 1, wherein the preserving agent is phenoxyethanol, potassium sorbate, benzyl alcohol, methyl paraben, chlorhexidine digluconate, chloroxylenol, chlorphenesin, dehydroacetic acid, diazolidinyl urea, DMDM hydantoin, ethylparaben, iodopropynyl butylcarbamate, methylisothiazolinone, propyllparaben, phenoxyethanol, phenoxyisopropanol, polyaminopropyl biguianide, sodium benzoate, salicylic acid, or any combination thereof.

6. The topically applicable composition of claim 1, wherein the composition comprises about 0.1% to about 0.8% by weight of the preserving agent.

7. The topically applicable composition of claim 1 further comprising a surfactant selected from the group consisting of docusate sodium (diethylhexyl sodium sulfosuccinate), poloxamer, PEG ether, PPG ester, alkyl polyglucoside, sorbitan ester, ethoxylated sorbitan ester, alcohol sulfate, ethoxylated alcohol sulfate, alkyl benzene sulfonate, alpha olefin sulfonate, sulfosuccinate, isethionate ester, taurate, alkyl betaine, alkyl amidopropyl betaine, amphoacetate, and alkyl sultaine.

8. The topically applicable composition of claim 1 further comprising a humectant.

9. The topically applicable composition of claim 1 further comprising a liquid wetting surfactant.

10. The topically applicable composition of claim 1 further comprising a gelling agent selected from the group consisting of acrylamide, sodium acryloyldimethyl taurate copolymer, isohexadecane, polysorbate 80, hydroxyethyl acrylate, and polysorbate 60.

11. The topically applicable composition of claim 1 further comprising a pro-penetrating agent selected from the group consisting of propylene glycol, dipropylene glycol, propylene glycol dipelargonate, lauroglycol, an alcohol, an alkylmethyl sulfoxide, a polyol, oleic acid, isopropyl myristate, decylmethyl sulfoxide, DMSO, urea, and ethoxydiglycol.

12. The topically applicable composition of claim 1 further comprising a sequestering agent selected from the group consisting of disodium ethylenediaminetetraacetic acid (EDTA), dihydroxyethylglycine, citric acid, and tartaric acid or a derivative or salt thereof.

13. The topically applicable composition of claim 1 further comprising a keratolytic agent, an oil, an antioxidant, a skin conditioning agent, or any combination thereof.

14. The topically applicable composition of claim 1, wherein the opacifer is titanium dioxide, the preserving agent is phenoxyethanol, and the composition further comprises docusate sodium (diethylhexyl sodium sulfosuccinate) as a surfactant; glycerol as a humectant; poloxamer 124 as a liquid wetting surfactant; acrylamide/sodium acryloyldimethyl taurate copolymer and isohexadecane and polysorbate 80 as a gelling agent; propylene glycol as a pro-penetrating agent; disodium EDTA as a sequestering agent; and purified water as a vehicle.

15. The topically applicable composition of claim 14 comprising:
i) about 3% by weight of the Isopropylcarbonate Benzoyl Peroxide;
ii) about 0.4% by weight of the titanium dioxide;
iii) about 0.4% by weight of the phenoxyethanol;
iv) about 0.05% by weight of the docusate sodium (diethylhexyl sodium sulfosuccinate) as a surfactant;
v) about 4% by weight of the glycerol;
vi) about 0.2% by weight of the poloxamer 124;
vii) about 4% by weight of the acrylamide/sodium acryloyldimethyl taurate copolymer and isohexadecane and polysorbate 80;
viii) about 4% by weight of the propylene glycol; and
ix) about 0.1% or about 0.2% by weight of the disodium EDTA.

16. A method for producing an Isopropylcarbonate Benzoyl Peroxide aqueous gel, which method comprises the steps of:
solubilizing a sequestering agent in aqueous solution to produce the main phase;
preparing a disaggregation medium comprising Isopropylcarbonate Benzoyl Peroxide by dispersing Isopropylcarbonate Benzoyl Peroxide, propylene glycol, glycerol and liquid wetting surfactant in water to produce medium A; and
adding titanium dioxide to the main phase;
adding a first portion of gelling agent to the main phase;
adding medium A to the main phase;
adding of phenoxyethanol to the main phase;
solubilizing docusate sodium in water to provide solubilized docusate sodium followed by addition of the solubilized docusate sodium to the main phase by gentle agitation; and
adding a second portion of gelling agent to the main phase,
whereby a gel is formed.

17. A method for treating common acne, comedones, polymorphous acne, nodulocystic acne, acne conglobata, or secondary acne afflicting the skin of an individual in need of such treatment, comprising topically administering to the individual a topically applicable composition according to claim 1.

18. A regimen for treatment of acne vulgaris, comprising,
cleaning skin; and
applying a topically applicable composition according to claim 1 to the skin.

19. A kit comprising
a) a topically applicable composition according to claim 1; and
b) a topical cleanser and/or a topical moisturizer.

20. The kit of claim 19, wherein the facial cleaner and/or the facial moisturizer comprise salicylic acid, benzoyl peroxide, or adapalene.
